SEQUENCE LISTING (10 sequences)

SEQ ID NO: 1 - protein, 7 aa, Artificial Sequence
  Synthetic
LRRASLG

SEQ ID NO: 2 - protein, 6 aa, Artificial Sequence
  Synthetic
XRRXAX

SEQ ID NO: 3 - protein, 7 aa, Artificial Sequence
  Synthetic
LRRAALG

SEQ ID NO: 4 - protein, 11 aa, Artificial Sequence
  Synthetic
MNGRTGRRNAI

SEQ ID NO: 5 - protein, 76 aa, Homo sapiens
MTDVETTYADFIASGRTGRRNAIHDILVSSASGNSNELALKLAGLDINKTEGEEDAQRSSTEQSGEAQGEAAKSES

SEQ ID NO: 6 - protein, 78 aa, Homo sapiens
MRTDSSKMTDVESGVANFASSARAGRRNALPDIQSSAATDGTSDLPLKLEALSVKEDAKEKDEKTTQDQLEKPQNEEK

SEQ ID NO: 7 - protein, 76 aa, Homo sapiens
  Synthetic
MMEVESSYSDFISCDRTGRRNAVPDIQGDSEAVSVRKLAGDMGELALEGAEGQVEGSAPDKEAGNQPQSSDGTTSS

SEQ ID NO: 8 - protein, 20 aa, Artificial Sequence
  Synthetic
IASGRTGRRNAIHDILVSSA

SEQ ID NO: 9 - protein, 14 aa, Artificial Sequence
  Synthetic
IASGRTGRRNAIHD

SEQ ID NO: 10 - protein, 18 aa, Artificial Sequence
  Synthetic
YADFIASGRTGRRNAIHD

(19) United States
(12) Patent Application Publication
(10) Pub. No.: US 2005/0148073 A1
(43) Pub. Date: Jul. 7, 2005

(54) Induction of hepatocyte proliferation in vitro by inhibition of cell cycle inhibitors

(76) Inventors: Linda K. Hansen, Minneapolis, MN (US); John T. Fassett, Minneapolis, MN (US)

Correspondence Address: FISH & RICHARDSON P.C., PO BOX 1022, MINNEAPOLIS, MN, 55440-1022, US

(21) Appl. No.: 10/945,854

(22) Filed: Sep. 20, 2004

Related U.S. Application Data
(63) Continuation-in-part of application No. PCT/US03/08778, filed on Mar. 20, 2003.
(60) Provisional application No. 60/366,459, filed on Mar. 20, 2002.

(51) Int. Cl. C12N 5/08

(52) U.S. Cl. 435/370; 435/404

(57) ABSTRACT

The present invention provides methods of inducing proliferative and differentiative mammalian hepatocytes, or survival of differentiative mammalian hepatocytes, in vitro comprising contacting the hepatocytes with protein kinase A (PKA) inhibitor.

### CROSS-REFERENCE TO RELATED APPLICATIONS

This application is a continuation-in-part application of PCT/US2003/008778 filed on Mar. 20, 2003 and published in English as WO 03/080649, which claims priority to U.S. Application Ser. No. 60/366,459, filed on Mar. 20, 2002, which applications and publication are incorporated herein by reference.

### BACKGROUND OF THE INVENTION

Acute and chronic liver failure represents a major clinical challenge with few treatment options available. While liver transplantation is effective in the treatment of certain cases, the increasing shortage of organ donors along with an increase in the incidence of chronic liver disorders such as hepatitis C are creating a critical need for the development of alternative therapies. Innovative treatments utilizing isolated hepatocytes hold great potential in meeting this need. However, achieving sufficient cell mass to replace liver function by either extracorporeal devices or in vivo cell transplantation, combined with limited cell sources, will require the ability to expand hepatocyte cell populations without loss of function or viability. Such methods currently do not exist.

Because of the wide range of hepatocyte functions that comprise liver function, it is virtually impossible to replace this function with anything but hepatocytes themselves. Thus, hepatocytes are being extensively utilized in experimental models for the treatment of liver disease. Such treatments include the development of extracorporeal devices (Nyber 1993, Rozga 1993) that may provide sufficient temporary liver support for the patient to either survive to transplant or for the diseased liver to regenerate on its own. Additional cell transplantation models are being pursued that incorporate either direct injection of hepatocytes into the body (Matas 1976) or attachment of hepatocytes onto three-dimensional scaffolds for in vivo transplantation as an alternative to organ transplantation (Vacanti 1988, Hansen 1992).

In spite of extensive research in the development of cell-based therapies, successful long-term solutions have been elusive. A major reason for the lack of success continues to be the challenges of maintaining isolated hepatocytes in a viable and differentiated state. While certain culture conditions, particularly specific extracellular matrix (ECM) components, may facilitate enhanced differentiated phenotype, these conditions are unable to support hepatocyte proliferation. Another persistent limitation of in vitro hepatocyte applications is their limited proliferative capacity.

Hepatocytes in vivo possess a remarkable regenerative capacity; yet this potential is lost upon isolation and placement in culture. While innovative recipes for tissue culture medium have led to improvements in hepatocyte proliferation in vitro, most conditions that promote proliferation also lead to simultaneous loss of differentiated function. Use of immortalized cells lines is also an unappealing option, as such cells lines are generally derived from dedifferentiated hepatomas and lack sufficient differentiated function relative to normal hepatocytes, leading to reduced function when incorporated into extracorporeal devices (Nyberg 1994).

It is estimated that approximately 10% of the liver mass is required to adequately replace most liver functions. Thus, any treatment involving hepatocytes will require large amount of cells, generating an imperative need for improvements in both stem cell and adult hepatocyte proliferation and differentiation in vitro.

### SUMMARY OF THE INVENTION

The present invention provides a method of inducing proliferative and differentiative mammalian hepatocytes in vitro by contacting the hepatocytes with protein kinase A (PKA) inhibitor. Such inhibitors include any compound, peptide, nucleotide derivative, nucleoside derivative, polysaccharide, sugar or other substance that can inhibit the activity of protein kinase A. An example of an appropriate PKA inhibitor is the compound H89. Another example of a PKA inhibitor is a protein kinase A inhibitory peptide (PKI). Such PKI peptides are synthetic peptides with the amino acid sequence of the substrate binding region of protein kinase A. Thus they compete with the interaction between PKA and its substrates, specifically inhibiting protein kinase A activity.

A “proliferative” cell is a cell that is, or is capable, of dividing into two daughter cells. A cell that is “differentiative” is a cell that is performing the specific functions attributed to the adult cells of the tissue of origin. The term “inducing” is used herein to mean stimulating the cell to express a particular function or response, for example. Such a response may be proliferation; an example of a function that may be induced is a differentiated function that is typically exhibited by a mature hepatocyte.

The hepatocytes that can be used in -the present invention include primary hepatocytes or hepatocytes generated from stem cells (either embryonic or adult stem cells). Primary hepatocytes are cells obtained directly from tissue, and are not immortalized. Such primary cells can be obtained, for example, from a biopsy from a mammal. Alternatively, the primary hepatocytes can be obtained from a culture of stem cells (embryonic or adult) that have been directed to differentiate to the hepatocyte lineage under special culture conditions. See, e.g., U.S. Pat. No. 6,458,589.

The method can further include growing the hepatocytes on a biocompatible support matrix. For example, the support matrix can be a collagen matrix, a collagen film or a collagen gel sandwich. Collagen can be produced into different types, or conformations, of matrix. One example is a film, which is a thin layer of collagen adsorbed onto a plastic dish. Another collagen matrix is gel, in which collagen polymerizes from solution into fibrils. Gel can be layered on the bottom of a dish, which is commonly then referred to simply as a collagen gel. When gel is also layered over the top of cells, it is called a collagen gel sandwich. A collagen gel sandwich is a matrix condition consisting of two layers of type I collagen, one on the basal surface of cells and one overlaying the upper surface of cells. The bottom layer is generally coated on a plate (usually type I collagen, or Matrigel™ or fibronectin) on which cells are plated. After cell attachment, a type I collagen solution is poured over the cells and allowed to polymerize.

The hepatocytes used in the present invention can be of mammalian origin, for example, of human or porcine origin. In the present invention, the hepatocytes are contacted with PKA inhibitor for a period of time, or for intervals of time. For example, in one embodiment, the hepatocytes are in contact with PKA inhibitor for a period of time, followed by a period of time in which they are allowed to grow in the absence of PKA inhibitor. This process can be repeated by one or more other cycles of being in contact with PKA inhibitor followed by a period of growing in the absence of PKA inhibitor. This cyclical process can be referred to as “pulsing” the hepatocytes with PKA inhibitor. In certain embodiments, such pulsing increases the number of cells present in the growth chamber, matrix, dish, etc. For example, the number of cells can increase by more than about 1%, 5%, 10%, 15%, 20%, 50%, 80%, 90% or 100%, in each pulsing cycle.

The PKA inhibitor is present at a concentration of about 0.1 to about 10 μM, or even at about 0.5 to 5 μM. For example, the concentration may be at about 0.1, 0.5,1, 3, or 5 μM.

The present invention also provides a method of screening a compound for its effect on hepatocytes or a hepatocyte activity by combining the compound with a cell population obtained by treating hepatocytes with a PKA inhibitor; determining any change to cells in the population or their activity that results from being combined with the compound; and correlating the change with the effect of the compound on hepatocytes or a hepatocyte. The method can further involve determining whether the compound is toxic to cells in the population; determining whether the compound affects ability of cells in the population to proliferate or be maintained in culture; determining whether the compound changes enzyme activity or secretion; determining whether the compound changes activity of a hepatocyte Phase I metabolizing enzyme; determining whether the compound changes activity of a hepatocyte Phase II metabolizing enzyme; determining whether the compound changes cytochrome p450 expression or activity; determining whether the compound changes CYP3A3-5 activity, CYP2D activity, or CYP2C9 activity; determining whether the compound affects CYP1A1 or CYP1A2 activity; determining whether the compound affects the activity of 7-ethoxycoumarin O-de-ethylase, aloxyresorufin O-de-alkylase, coumarin 7-hydroxylase, p-nitrophenol hydroxylase, testosterone hydroxylation, UDP-glucuronyltransferase, glutathione S-transferase, gamma-glutamyl transpeptidase, or glucose-6-phosphatase; and/or determining whether the compound affects the synthesis of a plasma protein (such as albumin, transferrin, alpha₁-antitrypsin (AAT), or alpha-fetoprotein).

The present method further involves determining whether the compound affects gluconeogenesis, ureagenesis, bilirubin conjugation, or bile acid conjugation; and/or determining whether the compound affects synthesis or secretion of cholesterol or lipoprotein, the level of glutathione, nucleoside phosphate metabolism, intracellular K²⁺ or Ca⁺ concentration, the release of nuclear matrix proteins or oligonucleosomes, induction of apoptosis, or glycogen storage. The hepatocytes used in these methods can be mammalian hepatocytes; in some embodiments the hepatocytes are human in origin. Further, they can be genetically altered cells.

The present invention further provides a bioartificial liver device including a proliferative and differentiative mammalian hepatocyte. This device can further include a biocompatible support matrix (e.g., a gel matrix, a film or a collagen gel sandwich).

The present invention also provides an artificial liver device including a cell culture layer including an isolated cell line of normal hepatocytes and a PKA inhibitor, and a support matrix that provides means for fluid circulation across the cell culture layer. Examples of suitable fluids are culture medium, plasma or blood.

The hepatocytes of the invention can be grown or maintained in a growth environment. A “growth environment” is an environment in which cells of interest will proliferate in vitro. Features of the environment include the medium in which the cells are cultured, the temperature, the partial pressure of O₂ and CO₂, and a supporting structure (such as a substrate on a solid surface) if present. The medium in which the cells are cultured can contain an inhibitor of protein kinase A, as provided by the invention, to induce proliferation while maintaining differentiation. The medium can be a nutrient medium. A “nutrient medium” is a medium for culturing cells containing nutrients that promote proliferation. The nutrient medium can contain a protein kinase inhibitor as indicated by the invention. The nutrient medium may also contain any of the following in an appropriate combination: isotonic saline, buffer, amino acids, antibiotics, serum or serum replacement, and exogenously added factors. A “conditioned medium” is prepared by culturing a first population of cells in a medium, and then harvesting the medium. The conditioned medium (along with anything secreted into the medium by the cells) may then be used to support the growth of a second population of cells.

The present invention provides a method of transplanting hepatocytes involving inducing a population of proliferative and differentiative mammalian hepatocytes in vitro comprising contacting the hepatocytes with protein kinase A (PKA) inhibitor; and transplanting the population of hepatocytes.

### BRIEF DESCRIPTION OF THE FIGURES

FIG. 1. Effects of collagen gel overlay on cell cycle progression on collagen film. Hepatocytes were cultured on collagen film, then transfected from 6-12 hr after plating with cyclin D1 promoter luciferase construct. At 12 hours, transfection medium was removed and collagen gel added. Promoter activity was assessed 72 hours after plating. Activity of an albumin promoter construct was simultaneously assessed. Data presented is the mean±SD of 5 (cyclin D1) and 2 (alb) experiments run in duplicate or triplicate.

FIG. 2. Effects of PKA inhibition on morphology and cell cycle progression on collagen gel. (A) PKA activity was assessed in hepatocytes cultured on gel or film. In vitro kinase assay was performed; DPM=total ³²P-incorporation, minus counts from sample without substrate. (B) DNA synthesis was assessed in hepatocytes cultured on collagen film or gel with H89 (2 μM) or DMSO. [³H]Thymidine incorporation was assessed from 48-96 hr. (C) Phosphorylation of p42/44 ERKs was determined in hepatocytes cultured on collagen gel for 50 hr by Western blot using a phospho-specific p42/44 antibody (NEB). (D) Hepatocytes were cultured on type I collagen film or gel for 9 hours in the presence or absence of H89 (3 μM) or the corresponding volume of DMSO (vehicle). EGF receptor (EGFr) was immunoprecipitated, followed by Western blot analysis of phospho-tyrosine (P-Tyr) or total EGFr.

FIG. 3. Scanning electron micrographs of hepatocytes cultured on collagen film or gel for 24 hours with PKA agonist (8-Br-cAMP) or inhibitor (H89). (A) Collagen film, (B) Collagen gel, (C) Collagen film, 8-Br-cAMP, and (D) Collagen gel, H89.

FIG. 4. Effects of PKA inhibition on hepatocyte proliferation and differentiation. (A) Hepatocytes were cultured on collagen gel in the presence of H89 (3 μM) or equivalent volume of DMSO (control) for 96 hr, at which time cells were trypsinized and counted by hemocytometer. (B) Hepatocytes were cultured on collagen film or gel with H89 (3 μM) or DMSO. Medium was replaced at 96 hr and collected for analysis at 120 hr. Albumin secretion was determined by ELISA. (C) Urea production was determined in hepatocytes cultured on collagen gel with or without H89. +NH₄ indicates addition of exogenous NH₄.

FIG. 5. (A) Enhanced hepatocyte number and maintenance of differentiated function by PKA inhibition (representative experiment). (B) Enhanced hepatocyte number by PKA inhibition (% increase) showing an average of five experiments.

FIG. 6. Dot blot of albumin secretion—72 hour media samples.

### DETAILED DESCRIPTION OF THE INVENTION

Acute and chronic liver failure represents a major clinical challenge with few treatment options available. Currently, the only viable option for treating chronic liver diseases or acute liver failure is liver transplantation. While liver transplantation is effective in the treatment of certain cases, the increasing shortage of organ donors along with an increase in the incidence of chronic liver disorders such as hepatitis C are creating a critical need for the development of alternative therapies.

According to the invention, hepatocytes treated or maintained according to the methods provided herein can be used for replacement of diseased liver tissues. In particular, hepatocytes can be replicated and maintained in vitro without significant loss of viability, proliferative capacity, and/or differentiated function.

Hepatocytes employed in the invention can be obtained from a variety of sources, including stem cells, hepatocyte precursor cells or differentiated hepatocytes. When undifferentiated stem cells are used, those stem cells should possess unlimited proliferative capacity to allow for repeated cell expansion, while also having differentiative capacity to allow for fully differentiation into functional hepatocytes. Recent studies have identified a multipotent adult progenitor cell (MAPC) from bone marrow that differentiates into hepatocyte-like cells, possessing comparable differentiated functions like those of adult hepatocytes (Schwartz, 2002; Jiang, 2002). While these stem cells hold enormous potential as a cell source for cell-based therapies, these cells typically lost their proliferative ability after becoming differentiated into hepatocyte-like cells when cultured under previously available conditions.

However, the invention has solved this problem. The invention provides methods for maintaining, manipulating and propagating hepatocytes, hepatocyte precursor cells or hepatocyte stem cells without loss of differentiated function that involve culturing the hepatocytes, hepatocyte precursor cells or hepatocyte stem cells in the presence of a protein kinase A inhibitor. The present inventors have performed experiments illustrating the mechanism of growth arrest on the differentiation-promoting substrates, elucidating growth-specific signaling events inhibited in hepatocytes cultured on type I collagen gel compared to those on a thin film of type I collagen, which promotes proliferation. These studies have determined that protein kinase A is induced upon interaction with type I collagen gel but not on collagen film. This elevated PKA activity specifically inhibits epidermal growth factor (EGF)-dependent pathways. The inventors have also found that inhibition of PKA activity restores specific G1 events and DNA synthesis on collagen gel. Furthermore, it was found that inhibition of PKA increased hepatocyte proliferation by 67% on collagen gel without loss of differentiated function (see Examples, below). These data suggest for the first time that identification and manipulation of specific signaling events may allow the propagation of differentiated hepatocytes in vitro. These studies show that manipulation of signaling pathways in primary hepatocytes or MAPCs maintained on specific ECM substrates permits proliferation while maintaining differentiation in long-term cultures.

In summary, primary hepatocytes have great potential for use in cell-based clinical therapies for the treatment of liver failure. Once hepatocytes are dissociated from liver tissue, however, they can lose both differentiative function and proliferative capacity. Culturing cells on certain extracellular matrix conditions, such as a collagen gel, can improve differentiated function in vitro but often at the loss of proliferative capacity. The inventors have determined that a specific inhibitor of cell proliferation, PKA, is active on the growth-inhibitory substrate, collagen gel, and that inhibiting PKA activity with a specific inhibitor of PKA, will allow cells to progress through the cell cycle on collagen gel. This discovery allows expansion of hepatocyte populations in vitro under conditions that simultaneously promote highly differentiated function. Previously, such conditions were used to support functional differentiated hepatocytes but were not capable of allowing cell proliferation.

PKA Inhibitors Promote Differentiated Hepatocyte Proliferation

Hepatocytes exist in the liver in a complex three-dimensional, polarized structure, characterized by extensive cell-cell and cell-extracellular matrix interactions. Cell membranes are polarized into basal, lateral, and apical (bile canalicular) domains that each maintain a specific set of functions. When hepatocytes are enzymatically digested away from the intact liver structure and placed in culture, a dramatic change in morphology occurs with concomitant loss of polarized structure and differentiated function. Alteration of culture conditions, most importantly the adhesive substrate, can improve retention of differentiated functions. Such adhesive conditions include gel-like substrates, such as type I collagen gel or Matrigel™, a substrate secreted by a sarcoma cell line.

However, the ability to maintain differentiated function has previously appeared to be inversely correlated with proliferative potential, such that conditions that promote differentiated function result in a loss of proliferative capacity, and vice versa. While tissue culture media compositions have been developed that increase the proliferative capacity of hepatocytes in vitro (Block, 1996), the conditions that promote proliferation were previously accompanied by a significant drop in both differentiated function and/or long-term viability. On the other hand, cultures maintained on certain extracellular matrix substrates, such as collagen gel or Matrigel™, demonstrate enhanced function and viability, yet the proliferative capacity of these cells was consistently lost on these substrates. Thus, it has previously been impossible to achieve both expansion (i.e., proliferation) and differentiated function (i.e., differentiation) of adult hepatocytes under the same conditions in vitro.

The invention solves this problem by providing compositions comprising inhibitors of protein kinase A and methods of using such inhibitors that promote proliferation of hepatocytes without loss of the desirable differentiated functions associated with mature hepatocytes. Such inhibitors include any compound, peptide, nucleotide derivative, nucleoside derivative, polysaccharide, sugar or other substance that can inhibit the activity of protein kinase A.

One of skill in the art can select useful protein kinase A inhibitors by observing the activity of protein kinase A when exposed to an inhibitor either in vitro or in vivo. Protein kinase A activity, and inhibition of that activity, can be determined as the difference between phosphorylation of a PKA specific substrate with and without a specific PKA inhibitor present. The specific PKA substrate can be any convenient peptide with a serine that is recognized as a phosphorylation site by PKA. For example, the peptide substrate can have the sequence: Leu Arg Arg Ala Ser Leu Gly (SEQ ID NO:1).

Many PKA inhibitors are available and may be used. For example, many examples of PKA inhibitors including chemical structures, methods for administration and pharmacological effects are listed at the Calbiochem website at calbiochem.com. In general, inhibitors that also significantly inhibit protein kinase C activity are avoided.

In some embodiments, the protein kinase A inhibitor is a nucleotide or nucleoside derivative. For example, the inhibitor can have formula I: 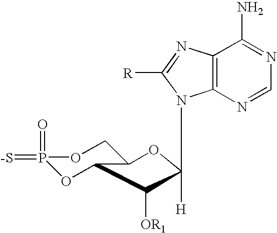
wherein R is hydrogen, halogen, or heterocycloalkyl and R₁ is hydrogen, lower alkyl, or lower acyl.

The following general definitions are used, unless otherwise described: halo is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to.

More specifically, lower alkyl means (C₁-C₆) alkyl. Such (C₁-C₆) alkyl can be methyl, ethyl, propyl, isopropyl, butyl, iso-butyl, sec-butyl, pentyl, 3-pentyl, or hexyl. Preferred lower alkyl groups are (C₁-C₃) alkyl including methyl ethyl, propyl, isopropyl and the like. Lower acyl refers to a carbonyl group attached to a lower alkyl group (e.g., —CO—CH₃).

Lower cycloalkyl generally means (C₃-C₆)cycloalkyl. Such (C₃-C₆)cycloalkyl can be cyclopropyl, cyclobutyl, cyclopentyl, or cyclohexyl. In some case the lower cycloalkyl can have substituents, for example, lower alkyl groups. Such alkyl-substituted lower cycloalkyl groups can, for example, be (C₃-C₆)cycloalkyl(C₁-C₆)alkyl groups. These (C₃-C₆)cycloalkyl(C₁-C₆)alkyl groups can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl.

Heterocycloalkyl encompasses a radical attached via a ring carbon of a monocyclic ring containing four to six ring atoms consisting of carbon and one to four heteroatoms each selected from the group consisting of non-peroxide oxygen, sulfur, and N(X) wherein X is absent or is H, O, (C₁-C₄)alkyl. Examples of heterocycloalkyl groups include piperidine, pyrrolidine, ethyleneimine, morpholine, tetrahydrofuran and the like.

Specific examples of nucleoside or nucleotide derivatives that act as protein kinase A inhibitors and that can be utilized in the invention can be found at the Calbiochem website (calbiochem.com). One such inhibitor is adenosine 3′,5′ cyclic monophosphorothioate, 2′-O-monobutyryl-, Rp-Isomer, sodium salt, depicted below.

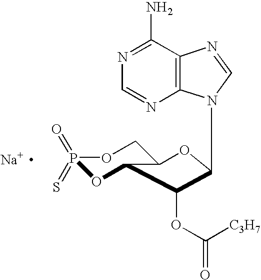

Another inhibitor that can be used is adenosine 3′,5′ cyclic monophosphorothioate, 8-chloro-, Rp-isomer, sodium salt, depicted below.

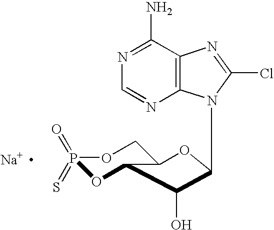

Another inhibitor that can be used is adenosine 3′,5′ cyclic monophosphorothioate, 8-piperidino-, Rp-isomer, sodium salt, depicted below.

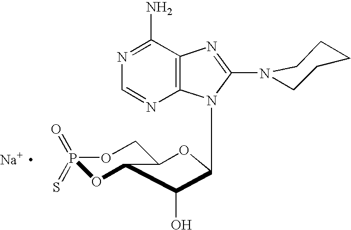

Another inhibitor that can be used is adenosine 3′,5′ cyclic monophosphorothioate, Rp-isomer, triethylammonium salt, depicted below.

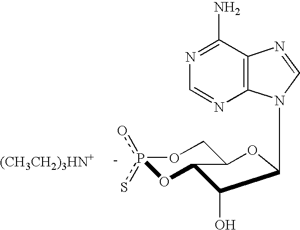

The H-89 inhibitor is a potent inhibitor of protein kinase A that can be used in the invention. The chemical name for the H-89 inhibitor is N-[2-((p-bromocinnamyl)amino)ethyl]-5-isoquinolinesulfonamide; it is available from Calbiochem. The structure of the H-89 dihydrochloride salt is given below.

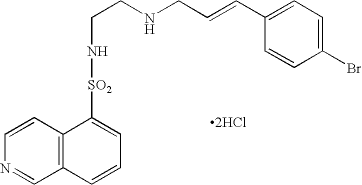

The KT5720 inhibitor from Calbiochem can also be used in the invention. The structure for this inhibitor is provided below.

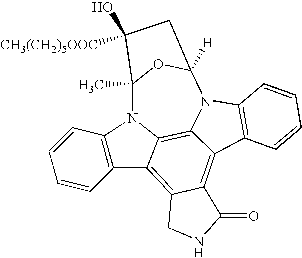

Ellagic acid is another inhibitor that can be used in the invention and that is available form Calbiochem. The chemical name for ellagic acid is 4,4′,5,5′,6,6′-hexahydroxydiphenic acid 2,6,2′,6′-dilactone, and the structure for this inhibitor is given below.

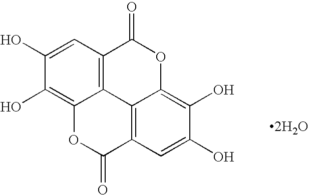

The protein kinase A inhibitor piceatannol, whose structure is shown below can also be used in the invention. Piceatannol is available from Calbiochem.

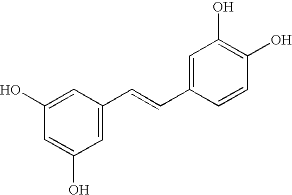

Another example of a compound that can be used as a protein kinase inhibitor is 1-(5-Isoquinolinesulfonyl)-2-methylpiperazine (H-7), which is available from Calbiochem. The dihydrochloride salt of this compound is shown below.

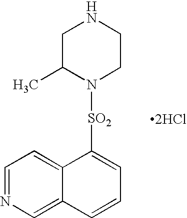

Another example of a compound that can be used as a protein kinase inhibitor is N-[2-(methylamino)ethyl]-5-isoquinolinesulfonamide (H-8), which is available from Calbiochem. The dihydrochloride salt of this compound is shown below.

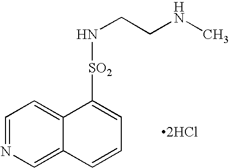

Another example of a compound that can be used as a protein kinase inhibitor is N-(2-aminoethyl)-5-isoquinolinesulfonamide (H-9), which is available from Calbiochem. The dihydrochloride salt of this compound is shown below.

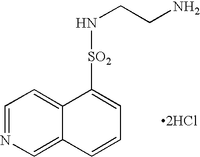

Another example of a compound that can be used as a protein kinase inhibitor is (5-isoquinolinesulfonyl)piperazine, 2HCl (H-100), which is available from Calbiochem. The dihydrochloride salt of this compound is shown below.

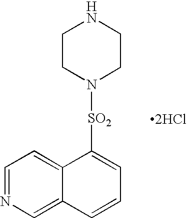

The PKA inhibitor can also be a peptide inhibitor (PKI). Such a peptide inhibitor can be any peptide that is recognized and bound by protein kinase A but that protein kinase A cannot phosphorylate. An example of a peptide inhibitor is a peptide with a “consensus sequence” for protein kinase A recognition but with alanine in place of serine, for example, a peptide with the following sequence:

| | |
|---|---|
| Xaa Arg Arg Xaa Ala Xaa | (SEQ ID NO:2) |

wherein Xaa is any amino acid, which specifically binds to the pseudoregion of the regulatory domain of PKA. Another example of such a peptide is a SEQ ID NO:1 peptide variant with alanine in place of serine, that is, a peptide with the following sequence:

| | |
|---|---|
| Leu Arg Arg Ala Ala Leu Gly | (SEQ ID NO:3) |

Myristoylated protein kinase A inhibitor amide (14-22, Cell-Permeable) having the sequence Myr-N-Gly-Arg-Thr-Gly-Arg-Arg-Asn-Ala-Ile-NH₂ (SEQ ID NO:4) is another example of a peptide inhibitor that can be utilized in the invention.

A variety of other PKI peptides can be used as an inhibitor of protein kinase A in the practice of the invention. For example, several PKI peptides can be found in the NCBI protein database. See website at ncbi.nlm.nih.gov/Genbank/GenbankOverview. One example of a human PKI peptide can be found at Genbank Accession No. P04541 (gi 417194)(SEQ ID NO:5), as follows:

| | | | | |
|---|---|---|---|---|
| 1 | MTDVETTYAD | FIASGRTGRR | NAIHDILVSS | ASGNSNELAL |
| 41 | KLAGLDINKT | EGEEDAQRSS | TEQSGEAQGE | AAKSES |

Another example of a human PKI peptide is at Genbank Accession No. Q9C010 (gi 17378640)(SEQ ID NO:6), as follows:

| | | | | |
|---|---|---|---|---|
| 1 | MRTDSSKMTD | VESGVANFAS | SARAGRRNAL | PDIQSSAATD |
| 41 | GTSDLPLKLE | ALSVKEDAKE | KDEKTTQDQL | EKPQNEEK |

Another example of a human PKI peptide is at Genbank Accession No. NP 008997 (gi 5902020)(SEQ ID NO:7, as follows:

| | | | | |
|---|---|---|---|---|
| 1 | MMEVESSYSD | FISCDRTGRR | NAVPDIQGDS | EAVSVRKLAG |
| 41 | DMGELALEGA | EGQVEGSAPD | KEAGNQPQSS | DGTTSS |

Another PKI that can be used as an inhibitor has the following sequence: Ile-Ala-Ser-Gly-Arg-Thr-Gly-Arg-Arg-Asn-Ala-Ile-His-Asp-Ile-Leu-Val-Ser-Ser-Ala (SEQ ID NO:8). Alternatively, the PKI to be used as an inhibitor with the following sequence: Ile-Ala-Ser-Gly-Arg-Thr-Gly-Arg-Arg-Asn-Ala-Ile-His-Asp (SEQ ID NO:9). In other embodiments, a PKI with the following sequence can be used as an inhibitor: Tyr-Ala-Asp-Phe-Ile-Ala-Ser-Gly-Arg-Thr-Gly-Arg-Arg-Asn-Ala-Ile-His-Asp (SEQ ID NO:10). See, Scott et al. Primary-structure requirements for inhibition by the heat-stable inhibitor of the cAMP-dependent protein kinase. Proc Natl Acad Sci U S A. March 1986;83(6):1613-16, for further details on this inhibitor.

Further examples of protein kinase A inhibitors are provided in the following references: Muniz et al., Proceedings of the National Academy of Sciences USA Dec. 23, 1997; 94(26) 14461-66; Baude et al., Journal of Biological Chemistry Vol. 269 issue 27 18128-18133 (July 1994); Scott et al.

Primary-structure requirements for inhibition by the heat-stable inhibitor of the cAMP-dependent protein kinase. Proc Natl Acad Sci U S A. March 1986;83(6):1613-16; and Scott et al., Identification of an inhibitory region of the heat-stable protein inhibitor of the cAMP-dependent protein kinase. Proc Natl Acad Sci U S A. July 1985;82(13):4379-83.

Culture Conditions

The hepatocytes employed in the compositions and methods of the present invention are isolated as described herein and maintained or cultured in an appropriate fluid, at an appropriate temperature and with exposure to an appropriate atmosphere. Examples of suitable fluids for maintaining, culturing and growing hepatocytes include culture medium, plasma or blood. A “growth environment” is an environment in which cells of interest will proliferate in vitro. A “nutrient medium” is a medium for culturing cells containing nutrients that promote proliferation. The nutrient medium may contain any of the following in an appropriate combination: isotonic saline, buffer, amino acids, antibiotics, serum or serum replacement, and exogenously added factors. A “conditioned medium” is prepared by culturing a first population of cells in a medium, and then harvesting the medium. The conditioned medium (along with anything secreted into the medium by the cells) may then be used to support the growth of a second population of cells.

In some embodiments, the culture media employed is Williams E media, which can be purchased from Sigma or Gibco-BRL. Such a culture media can be supplemented with additional factors including, for example, pyruvate at about 1 mM (available from Gibco-BRL), Insulin at about 20 mU/ml (available from Sigma), antibiotic such as penicillin and/or streptomycin at about 100 U/ml (available from Gibco-BRL), a buffer such as HEPES at about 20 mM (available from Gibco-BRL), steroids or hormones such as dexamethosone at about 5 nM (available from Sigma), vitamins such as ascorbic acid at about 100 ug/ml (available from Gibco-BRL), and growth factors such as endothelial growth factor or epithelial growth factor at 10 ng/ml (available from BD Biosciences).

Additional factors have been employed in the culture media to promote differentiated function. For example, addition of DMSO to cultures enhances certain differentiated functions. The formation of hepatocyte aggregates, or spheroids, which occurs in low-adhesive or non-adhesive conditions such as spinner cultures also leads to enhanced differentiated functions. Cyclin D1, a crucial cell cycle regulatory protein important for promoting progression from G1 into S phase of the cell cycle, appears to be a critical determinant of hepatocyte proliferation and its expression is exquisitely regulated by a number of extracellular factors including growth factors (Albrecht 1999), ECM (Hansen 1999), and cell-cell interaction. Studies indicate that over-expression of cyclin D1 can force growth-arrested hepatocytes on collagen gel to proceed at least through G1 into an S phase of the cell cycle (Nelsen 2001a). Indeed, over-expression of cyclin D1 in vivo can induce resting hepatocytes in the liver to undergo proliferation resulting in increased liver mass (Nelson 2001b). These conditions can also be employed in the practice of the invention.

Other features of the environment can beneficially influence the growth and maintenance of hepatocytes in culture including the temperature, the partial pressure of O₂ and CO₂, and a supporting structure (such as a substrate on a solid surface) if present. For example, the support matrix can be a collagen matrix, a collagen film or a collagen gel sandwich. Collagen can be produced into different types, or conformations, of matrix. One example is a film, which is a thin layer of collagen adsorbed onto a plastic dish. Another collagen matrix is gel, in which collagen polymerizes into fibrils. Gel can be layered on the bottom of a dish, which is commonly then referred to simply as a collagen gel. When gel is also layered over the top of cells, it is called a collagen gel sandwich. A collagen gel sandwich is a matrix condition consisting of two layers of type I collagen, one on the basal surface of cells and one overlaying the upper surface of cells. The bottom layer is generally coated on a plate (usually type I collagen, or Matrigel™ or fibronectin) on which cells are plated. After cell attachment, a type I collagen solution is poured over the cells and allowed to polymerize.

Hepatocyte Cells

The hepatocytes used in the invention can be obtained from a variety of sources and can be at different stages of differentiation. In general, while mature, differentiated hepatocytes are preferred for certain applications, younger, hepatocyte precursors are also useful for other applications. Moreover, when a source of hepatocyte stem cells is available, those stem cells can be cultured under conditions that promote the growth and differentiation of mature hepatocytes. Hence, hepatocyte precursor cells and hepatocyte stem cells are highly useful in the invention even when mature hepatocytes are ultimately chosen to be employed in a specific application.

A “hepatocyte precursor cell” or a “hepatocyte stem cell” is a cell that can proliferate and further differentiate into a hepatocyte, under suitable environmental conditions. Such cells may on occasion have the capacity to produce other types of progeny, such as oval cells, bile duct epithelial cells, or additional hepatocyte precursor cells. In particular, two classes of progenitors in the liver have been identified that have “stem cell” characteristics: oval cells and peri-ductular endodermal stem cells (Petersen 1999, Sell 2001, Suzuki 2002, Suzuki 2000).

Prototype “Pluripotent Stem cells” (PS cells) are pluripotent cells derived from pre-embryonic, embryonic, or fetal tissue at any time after fertilization, and have the characteristic of being capable under the right conditions of producing progeny of several different cell types. As defined for the purposes of this disclosure, PS cells are capable of producing progeny that are derivatives of all of the three germinal layers: endoderm, mesoderm, and ectoderm, according to a standard art-accepted test, such as the ability to form a teratoma in a suitable host.

Non-limiting examples of pluripotent stem cells are human embryonic stem (hES) cells (Thomson 1998); embryonic stem cells from other primates, such as Rhesus stem cells (Thomson 1995); and human embryonic germ (hEG) cells (Shamblott 1998). Other types of non-malignant pluripotent cells are also included in the term. Specifically, any cells of primate origin that are fully pluripotent (capable of producing progeny that are derivatives of all three germinal layers) are included, regardless of whether they were derived from embryonic tissue, fetal tissue, or other sources. PS cell cultures are said to be “essentially undifferentiated” when they display the morphology that clearly distinguishes them from differentiated cells of embryo or adult origin. PS cells typically have high nuclear/cytoplasmic ratios, prominent nucleoli, and compact colony formation with poorly discernable cell junctions, and are easily recognized by those skilled in the art. Colonies of undifferentiated cells can be surrounded by neighboring cells that are differentiated. Nevertheless, the essentially undifferentiated colony will persist when cultured under appropriate conditions, and undifferentiated cells constitute a prominent proportion of cells proliferating upon passaging of the cultured cells. Cell populations that contain any proportion of undifferentiated PS with these criteria can be used in this invention. Cell cultures described as essentially undifferentiated will typically contain at least about 20%, 40%, 60%, or 80% undifferentiated PS, in order of increasing preference.

Until recently, it was thought that adult stem cells could only differentiate into cells of the tissue of origin. Several recent studies, though, suggested that adult stem cells differentiate into lineages other than the tissue of origin. Following transplantation of BM or enriched hematopoietic stem cells (HSC), skeletal myoblasts (Ferrari 1998, Gussoni 1999) cardiac myoblasts (Orlic 2001, Jackson 2001), endothelium ( Jackson 2001, Lin 2000), hepatic and biliary duct epithelium (Petersen 1999, Lagasse 2000, Theise 2000), and other tissue-specific cells of donor origin have been detected.

A rare cell within human BM mesenchymal stem cell (MSC) cultures that can be expanded for greater than 100 population doublings has been identified (WO 01/11011). This cell differentiates not only into mesenchymal lineage cells but also endothelium and endoderm. This cell was termed a “multipotent adult progenitor cell” or MAPC. Similar MAPCs can be generated from mouse and rat BM. Because MAPCs proliferate extensively without obvious senescence or loss of differentiation potential, differentiate into functional hepatocytes in vitro, and into lung epithelium and hepatic epithelium in vivo, they may be an ideal cell source for cell-based clinical therapies.

If mature hepatocytes are desired, liver cells can be obtained from a suitable mammalian source to generate primary cultures of mature hepatocytes. Alternatively, stem cells or hepatocyte precursor cells can be obtained and differentiated into mature hepatocytes. Cells can also be sorted for multiple markers that distinguish distinct subcategories of hepatic precursor cell populations. Examples of markers include (a) the extent of granularity as measured by side scatter on fluorescence activated cell sorting, wherein more immature cell populations are more agranular, and increasing granularity correlates with increasing maturity; (b) the extent of autofluorescence, wherein increasing autofluorescence correlates with increasing maturity; and/or (c) the expression of a hepatic cell marker (such as the oval cell marker OC.3, which is detected by monoclonal antibody 374.3).

Liver cells which do not express hemopoietic or endothelial cell antigens recognized by monoclonal antibodies OX-43 and/or OX-44 (which recognize myeloid cells and endothelia) and which do not express antigens recognized by a monoclonal antibody to an erythroid antigen comprise less mature hepatoblasts. Different hepatoblasts have different characteristics, as follows:
(1) More granular cells, which are OC.3⁺, are committed bile duct precursors. These cells are also AFP⁺, albumin⁺ and CK 19⁺.
(2) More granular cells, which are OC.3⁻, are committed hepatocyte precursors. These cells are also AFP⁺, albumin⁺⁺⁺, and CK 19⁻.
(3) Agranular cells, which are OC.3⁺, are very immature hepatic precursors. These cells are also AFP⁺⁺⁺, albumin⁺ and CK 19⁻.

The invention is further directed to the use of hepatocytes cultured by the methods of the invention. The isolated hepatocytes of the invention can be used for to treat liver dysfunction. For example, hepatocytes can be injected into the body, such as into the liver or into an ectopic site. Whole liver transplantation, which requires costly and dangerous major surgery, can be replaced by a minor surgical procedure which introduces hepatocytes either into the liver via the portal vein or at an ectopic site such as the spleen. In addition, hepatocytes can be used in bioreactors or in culture apparatus to form artificial livers. Further, hepatocytes can be used in gene therapy, drug testing, vaccine production and any research, commercial or therapeutic purpose that requires liver cells of varying extents of maturity. These utilities are further described below.

Applications

The ability to enhance the propagation and differentiation of hepatocytes facilitates the development of many different hepatocyte-based tissue engineering applications. The improved procedures for adult hepatocyte propagation provided herein can be utilized in several types of in vivo cell transplantation applications, including models in which a patient's own hepatocytes are harvested and expended for eventual autologous cell transplantation following in vitro gene therapy, or when cells are obtained from a single donated organ and expanded for multiple heterologous cell transplantation. Alternatively, stem cells may be propagated into differentiated hepatocytes and used in these models, or in bioartificial liver devices, offering an easily renewable and readily available source of cells for large scale reactor use.

In addition to tissue engineering applications, the development of a tissue culture system in which adult human hepatocytes are propagated and maintained long term in a differentiated state is extremely beneficial in pharmaceutical applications for drug metabolism and toxicity testing. Such in vitro tests in isolated hepatocytes are currently very difficult due to their rapid dedifferentiation and particularly the loss of cytochrome P450 enzyme expression and activity. Maintenance of cytochrome P450 enzyme activity on type I collagen substrates along with retained proliferative capacity would greatly benefit this field.

The present invention addresses a great need in tissue engineering, which is the limitation of cell sources and ability to propagate such cells in culture. This problem is particularly acute in the field of liver tissue engineering, in which the functional cell type, the hepatocyte, is stubbornly difficult to propagate and maintain in a differentiated state. The starting materials of the present invention can be stem cells, and also primary hepatocytes.

This invention provides a method by which large numbers of cells of the hepatocyte lineage can be produced. These cell populations can be used for a number of important research, development, and commercial purposes.

Restoration of Liver Function

The invention provides for the use of proliferative and differentiative hepatocytes to restore a degree of liver function to a subject needing such therapy, perhaps due to an acute, chronic, or inherited impairment of liver function.

To determine the suitability of proliferative and differentiative hepatocytes for therapeutic applications, the cells can first be tested in a suitable animal model. At one level, cells are assessed for their ability to survive and maintain their phenotype in vivo. Proliferative and differentiative hepatocytes are administered to immunodeficient animals (such as SCID mice, or animals rendered immunodeficient chemically or by irradiation) at a site amenable for further observation, such as under the kidney capsule, into the spleen, or into a liver lobule. Tissues are harvested after a period of a few days to several weeks or more, and assessed as to whether proliferative and differentiative hepatocytes are still present. This can be performed by providing the administered cells with a detectable label (such as green fluorescent protein, or beta-galactosidase); or by measuring a constitutive marker specific for the administered cells. Where proliferative and differentiative hepatocytes are being tested in a rodent model, the presence and phenotype of the administered cells can be assessed by immunohistochemistry or ELISA using human-specific antibody, or by RT-PCR analysis using primers and hybridization conditions that cause amplification to be specific for human polynucleotide sequences. General descriptions for determining the fate of hepatocyte-like cells in animal models is provided in Grompe (1999); Peeters (1997;) and Ohashi (2000).

At another level, proliferative and differentiative hepatocytes are assessed for their ability to restore liver function in an animal lacking full liver function. Braun (2000) outlines a model for toxin-induced liver disease in mice transgenic for the HSV tk gene. Rhim (1995) and Lieber (1995) outline models for liver disease by expression of urokinase. Mignon (1998) outline liver disease induced by antibody to the cell-surface marker Fas. Overturf (1998) have developed a model for Hereditary Tyrosinemia Type I in mice by targeted disruption of the Fah gene. The animals can be rescued from the deficiency by providing a supply of 2-(2-nitro-4-fluoro-methyl-benzyol)-1,3-cyclohexanedione (NTBC), but develop liver disease when NTBC is withdrawn. Acute liver disease can be modeled by 90% hepatectomy (Kobayashi 2000). Acute liver disease can also be modeled by treating animals with a hepatotoxin such as galactosamine, CCl₄, or thioacetamide. Chronic liver diseases such as cirrhosis can be modeled by treating animals with a sub-lethal dose of a hepatotoxin long enough to induce fibrosis (Rudolph 2000). Assessing the ability of differentiated cells to reconstitute liver function involves administering the cells to such animals, and then determining survival over a 1 to 8 week period or more, while monitoring the animals for progress of the condition. Effects on hepatic function can be determined by evaluating markers expressed in liver tissue, cytochrome p450 activity, and blood indicators, such as alkaline phosphatase activity, bilirubin conjugation, and prothrombin time), and survival of the host. Any improvement in survival, disease progression, or maintenance of hepatic function according to any of these criteria relates to effectiveness of the therapy, and can lead to further optimization.

This invention includes differentiated cells that are encapsulated, or part of a bioartificial liver device. Various forms of encapsulation are described in “Cell Encapsulation Technology and Therapeutics”, Kuhtreiber et al. eds., Birkhauser, Boston Mass., 1999. Differentiated cells of this invention can be encapsulated according to such methods for use either in vitro or in vivo.

Bioartificial organs for clinical use are designed to support an individual with impaired liver function—either as a part of long-term therapy, or to bridge the time between a fulminant hepatic failure and hepatic reconstitution or liver transplant. Bioartificial liver devices are reviewed by Macdonald et al., pp. 252-286 of “Cell Encapsulation Technology and Therapeutics”, op cit., and exemplified in U.S. Pat. Nos. 5,290,684, 5,624,840, 5,837,234, 5,853,717, and 5,935,849. Suspension-type bioartificial livers comprise cells suspended in plate dialyzers, or microencapsulated in a suitable substrate, or attached to microcarrier beads coated with extracellular matrix. Alternatively, hepatocytes can be placed on a solid support in a packed bed, in a multiplate flat bed, on a microchannel screen, or surrounding hollow fiber capillaries. The device has inlet and outlet through which the subject's blood is passed, and sometimes a separate set of ports for supplying nutrients to the cells.

Current proposals for such liver support devices involve hepatocytes from a xenogeneic source, such as a suspension of porcine hepatocytes, because of the paucity of available primary human hepatocytes. Xenogeneic tissue sources raise regulatory concerns regarding immunogenicity and possible cross-species viral transmission.

The present invention provides a system for generating preparative cultures of human cells. Differentiated pluripotent stem cells are prepared according to the methods described earlier, and then plated into the device on a suitable substrate, such as a matrix of Matrigel™ or collagen. The efficacy of the device can be assessed by comparing the composition of blood in the afferent channel with that in the efferent channel—in terms of metabolites removed from the afferent flow, and newly synthesized proteins in the efferent flow.

Devices of this kind can be used to detoxify a fluid such as blood, wherein the fluid comes into contact with the differentiated cells of this invention under conditions that permit the cell to remove or modify a toxin in the fluid. The detoxification will involve removing or altering at least one ligand, metabolite, or other compound (either natural or synthetic) that is usually processed by the liver. Such compounds include but are not limited to bilirubin, bile acids, urea, heme, lipoprotein, carbohydrates, transferrin, hemopexin, asialoglycoproteins, hormones like insulin and glucagon, and a variety of small molecule drugs. The device can also be used to enrich the efferent fluid with synthesized proteins such as albumin, acute phase reactants, and unloaded carrier proteins. The device can be optimized so that a variety of these functions are performed, thereby restoring as many hepatic functions as are needed. In the context of therapeutic care, the device processes blood flowing from a patient in hepatocyte failure, and then the blood is returned to the patient.

The present invention relates to a liver-assist device. Example of devices that are used for bioartificial liver support is found in U.S. Pat. No. 6,294,380; U.S. Pat. No. 5,866,420; U.S. Pat. No. 5,605,835; U.S. Pat. No. 5,595,909; U.S. Pat. No. 4,853,324 and U.S. Pat. No. 4,675,002. All of these patents are incorporated by reference herein.

An embodiment of the present invention is a novel detoxification system, referred to as a “specific Extracorporeal Liver Assist Device” (“sELAD”), which is based on a special functional hepatocyte cell line. By the term “specific Extracorporeal Liver Assist Device” it is meant an extracorporeal liver support system which provides augmentation of functional activities which are typically diminished in hepatic dysfunction, and which are considered important in the recovery from hepatic coma, frequently seen in fulminant hepatic failure patients. Since it is difficult to obtain an extracorporeal liver support system which displays all of the biochemical potential of hepatocytes in vivo, the system may possess some of the main hepatic functions, preferentially detoxification. Preferably the extracorporeal liver support system possesses enhanced or elevated detoxification function by way of employment of hepatocytes, inoculated into the system, which have several times higher the content of detoxification enzymes and detoxificants than freshly isolated or transformed hepatocytes.

A bioartificial liver support system that applies the principles of the present invention provides an effective alternative to treat patients with fulminant hepatic failure. The device and method of the present invention can be used not only in bridging the patient to orthotopic transplantation, but also in preventing the patient from developing encephalopathy.

The immediate objective of any extracorporeal liver-support system is to maintain a patient with fulminant hepatic failure until the patient's own liver regenerates, or to bridge the patient to orthotropic transplantation. Most liver support systems used today are directed to blood detoxification because detoxification is considered an essential requirement for an extracorporeal liver support system. The metabolism of toxic substances by living cells in a hepatocyte bioreactor reduces toxicity, thus producing beneficial effects for the recovery of patients.

The quantity of hepatocytes inoculated into a bioreactor is another important factor that influences the efficiency of current bioartificial liver support systems. Although the exact number of hepatocytes in a convention bioreactor is not known, it is generally accepted that the cell mass is in the order of 100-300 g extracorporeal support.

The number of cells in a single bioreactor of the present invention can be about 2.5-7.5 billion in magnitude, about 25-75 gram per single module. In order to accommodate such a huge number of cells, a special bioreactor configuration was designed. The cells were allowed to grow to confluence on microcarriers. The cell-attached microcarriers where then moved into a bioreactor, where a continuous supply of nutrition and oxygen was provided to guarantee maintenance of steady functioning of the hepatocytes.

In modulation of the detoxification function, it was determined that highly differentiated human hepatocytes are optimal. An immortalized cell line with highly differentiated functions is preferred. Besides providing an unlimited cell division capacity, immortalized human hepatocytes obviate concerns about species specific metabolic differences. Further any infusion of proteins from the human hepatocytes is less likely to cause immune-mediated reactions than non-human proteins, especially after prolonged or repeated use. Methods for the establishment of immortalized cell lines are well known in the art, and are available using advanced cell and tissue culture technology. However, in order to obtain a clonal expansion of hepatocytes from normal liver tissue, special procedures in developing cell line are necessary, such as the use of new type matrix, growth factors or conditioned medium and induction of clonal expansion.

Proliferative and differentiative hepatocytes of this invention that demonstrate desirable functional characteristics in animal models (such as those described above) may also be suitable for direct administration to human subjects with impaired liver function. For purposes of hemostasis, the cells can be administered at any site that has adequate access to the circulation, typically within the abdominal cavity. For some metabolic and detoxification functions, it is advantageous for the cells to have access to the biliary tract. Accordingly, the cells are administered near the liver (e.g., in the treatment of chronic liver disease) or the spleen (e.g., in the treatment of fulminant hepatic failure). In one method, the cells administered into the hepatic circulation either through the hepatic artery, or through the portal vein, by infusion through an in-dwelling catheter. A catheter in the portal vein can be manipulated so that the cells flow principally into the spleen, or the liver, or a combination of both. In another method, the cells are administered by placing a bolus in a cavity near the target organ, typically in an excipient or matrix that will keep the bolus in place. In another method, the cells are injected directly into a lobe of the liver or the spleen.

The differentiated cells of this invention can be used for therapy of any subject in need of having hepatic function restored or supplemented. Human conditions that may be appropriate for such therapy include fulminant hepatic failure due to any cause, viral hepatitis, drug-induced liver injury, cirrhosis, inherited hepatic insufficiency (such as Wilson's disease, Gilbert's syndrome, or alpha₁-antitrypsin deficiency), hepatobiliary carcinoma, autoimmune liver disease (such as autoimmune chronic hepatitis or primary biliary cirrhosis), and any other condition that results in impaired hepatic function. For human therapy, the dose is generally between about 10⁹ and 10¹² cells, and typically between about 5×10⁹ and 5×10¹⁰ cells, making adjustments for the body weight of the subject, nature and severity of the affliction, and the replicative capacity of the administered cells. The ultimate responsibility for determining the mode of treatment and the appropriate dose lies with the managing clinician.

Preparation of Expression Libraries and Specific Antibody

The differentiated cells of this invention can also be used to prepare a cDNA library relatively uncontaminated with cDNA preferentially expressed in cells from other lineages. For example, the cells are collected by centrifugation at 1000 rpm for 5 min, and then mRNA is prepared from the pellet by standard techniques (Sambrook 2001). After reverse transcribing into cDNA, the preparation can be subtracted with cDNA from any or all of the following cell types: undifferentiated PS, embryonic fibroblasts, visceral endoderm, sinusoidal endothelial cells, bile duct epithelium, or other cells of undesired specificity, thereby producing a select cDNA library, reflecting expression patterns that are representative of mature hepatocytes, hepatocyte precursors, or both.

The proliferative and differentiative hepatocytes of this invention can also be used to prepare antibodies that are specific for hepatocyte markers, progenitor cell markers, markers that are specific for hepatocyte precursors, and other antigens that may be expressed on the cells. The cells of this invention provide an improved way of raising such antibodies because they are relatively enriched for particular cell types compared with proliferative and differentiative hepatocyte cultures and hepatocyte cultures made from liver tissue. Polyclonal antibodies can be prepared by injecting a vertebrate with cells of this invention in an immunogenic form. Production of monoclonal antibodies is described in a standard reference such as Methods in Enzymology 73B:3 (1981), or U.S. Pat. Nos. 4,491,632, 4,472,500 and 4,444,887. Other methods of obtaining specific antibody molecules (optimally in the form of single-chain variable regions) involve contacting a library of immunocompetent cells or viral particles with the target antigen, and growing out positively selected clones (See Marks 1996, International Patent Applications WO 94/13804, WO 92/01047, WO 90/02809, and McGuiness 1996). By positively selecting using proliferative and differentiative hepatocytes of this invention, and negatively selecting using cells bearing more broadly distributed antigens (such as differentiated embryonic cells) or adult-derived stem cells, the desired specificity can be obtained. The antibodies in turn can be used to identify or rescue hepatocyte precursor cells of a desired phenotype from a mixed cell population, for purposes such as co-staining during immunodiagnosis using tissue samples, and isolating such cells from mature hepatocytes or cells of other lineages.

Genomics

Proliferative and differentiative hepatocytes are of interest to identify expression patterns of transcripts and newly synthesized proteins that are characteristic for hepatocyte precursor cells, and may assist in directing the differentiation pathway or facilitating interaction between cells. Expression patterns of the differentiated cells are obtained and compared with control cell lines, such as undifferentiated hepatocytes or PS cells, other types of committed precursor cells (such as PS cells differentiated towards other lineages, hematopoietic stem cells, precursor cells for other mesoderm-derived tissue, precursor cells for endothelium or bile duct epithelium, hepatocyte stem cells obtained from adult tissues, or PS cells differentiated towards the hepatocyte lineage using alternative reagents or techniques).

Suitable methods for comparing expression at the protein level include the immunoassay or immunohistochemistry techniques describe earlier. Suitable methods for comparing expression at the level of transcription include methods of differential display of mRNA (Liang 1992), and matrix array expression systems (Schena 1995; Eisen 1999; Brown 1999).

The use of microarray in analyzing gene expression is reviewed by several researchers (Fritz 2000; “Microarray Biochip Technology”, M. Schena ed., Eaton Publishing Company; “Microarray analysis”, Gwynne 1999); Pollack 1999; Gerhold 1999; “Gene Chips (DNA Microarrays)”, L Shi, www.Gene-Chips.com). Systems and reagents for performing microarray analysis are available commercially from companies such as Affymetrix, Inc., Santa Clara Calif.; Gene Logic Inc., Columbia Md.; Hyseq Inc., Sunnyvale Calif.; Molecular Dynamics Inc., Sunnyvale Calif.; Nanogen, San Diego Calif.; and Synteni Inc., Fremont Calif. (acquired by Incyte Genomics, Palo Alto Calif.).

Solid-phase arrays are manufactured by attaching the probe at specific sites either by synthesizing the probe at the desired position, or by presynthesizing the probe fragment and then attaching it to the solid support. A variety of solid supports can be used, including glasses, plastics, ceramics, metals, gels, membranes, paper, and beads of various compositions. U.S. Pat. No. 5,445,934 discloses a method of on-chip synthesis, in which a glass slide is derivatized with a chemical species containing a photo-cleavable protecting group. Each site is sequentially deprotected by irradiation through a mask, and then reacted with a DNA monomer containing a photoprotective group. Methods for attaching a presynthesized probe onto a solid support include adsorption, ultra violet linking, and covalent attachment. In one example, the solid support is modified to carry an active group, such as hydroxyl, carboxyl, amine, aldehyde, hydrazine, epoxide, bromoacetyl, maleimide, or thiol groups through which the probe is attached (U.S. Pat. Nos. 5,474,895 and 5,514,785).

The probing assay is typically conducted by contacting the array by a fluid potentially containing the nucleotide sequences of interest under suitable conditions for hybridization, and then determining any hybrid formed. For example, mRNA or DNA in the sample is amplified in the presence of nucleotides attached to a suitable label, such as the fluorescent labels Cy3 or Cy5. Conditions are adjusted so that hybridization occurs with precise complementary matches or with various degrees of homology, as appropriate. The array is then washed, and bound nucleic acid is determined by measuring the presence or amount of label associated with the solid phase. Different samples can be compared between arrays for relative levels of expression, optionally standardized using genes expressed in most cells of interest, such as a ribosomal or house-keeping gene, or as a proportion of total polynucleotide in the sample. Alternatively, samples from two or more different sources can be tested simultaneously on the same array, by preparing the amplified polynucleotide from each source with a different label.

An exemplary method is conducted using a Genetic Microsystems array generator, and an Axon GenePix™. Scanner. Microarrays are prepared by first amplifying cDNA fragments encoding marker sequences to be analyzed in a 96 or 384 well format. The cDNA is then spotted directly onto glass slides at a density as high as >5,000 per slide. To compare mRNA preparations from two cells of interest, one preparation is converted into Cy3-labeled cDNA, while the other is converted into Cy5-labeled cDNA. The two cDNA preparations are hybridized simultaneously to the microarray slide, and then washed to eliminate non-specific binding. Any given spot on the array will bind each of the cDNA products in proportion to abundance of the transcript in the two original mRNA preparations. The slide is then scanned at wavelengths appropriate for each of the labels, the resulting fluorescence is quantified, and the results are formatted to give an indication of the relative abundance of mRNA for each marker on the array.

Identifying expression products for use in characterizing and affecting differentiated cells of this invention involves analyzing the expression level of RNA, protein, or other gene product in a first cell type, such as a PS cell differentiated along the hepatocyte lineage, analyzing the expression level of the same product in a control cell type, comparing the relative expression level between the two cell types, (typically normalized by total protein or RNA in the sample, or in comparison with another gene product expected to be expressed at a similar level in both cell types, such as a house-keeping gene), and identifying products of interest based on the comparative expression level.

Products will typically be of interest if their relative expression level is at least about 2-fold, 10-fold, or 100-fold elevated (or suppressed) in proliferative and differentiative hepatocytes of this invention, in comparison with the control. This analysis can optionally be computer-assisted, by marking the expression level in each cell type on an independent axis, wherein the position of the mark relative to each axis is in accordance with the expression level in the respective cell, and then selecting a product of interest based on the position of the mark. Alternatively, the difference in expression between the first cell and the control cell can be represented on a color spectrum (for example, where yellow represents equivalent expression levels, red indicates augmented expression and blue represents suppressed expression). The product of interest can then be selected based on the color representing expression of one marker of interest, or based on a pattern of colors representing a plurality of markers.

Proliferative and Differentiative Hepatocytes for Drug Screening

Proliferative and differentiative hepatocytes of this invention can be used to screen for factors (such as solvents, small molecule drugs, peptides, polynucleotides, and the like) or environmental conditions (such as culture conditions or manipulation) that affect the characteristics of differentiated cells of the hepatocyte lineage.

In some applications, proliferative and differentiative hepatocytes are used to screen factors that promote maturation of cells along the hepatocyte lineage, or promote proliferation and maintenance of such cells in long-term culture. For example, candidate hepatocyte maturation factors or growth factors are tested by adding them to the cells in different wells, and then determining any phenotypic change that results, according to desirable criteria for further culture and use of the cells.

Particular screening applications of this invention relate to the testing of pharmaceutical compounds in drug research. (See generally, “In vitro Methods in Pharmaceutical Research”, Academic Press, 1997, and U.S. Pat. No. 5,030,015). In this invention, proliferative and differentiative hepatocytes play the role of test cells for standard drug screening and toxicity assays, as have been previously performed on hepatocyte cell lines or primary hepatocytes in short-term culture. Assessment of the activity of candidate pharmaceutical compounds generally involves combining the differentiated cells of this invention with the candidate compound, determining any change in the morphology, marker phenotype, or metabolic activity of the cells that is attributable to the compound (compared with untreated cells or cells treated with an inert compound), and then correlating the effect of the compound with the observed change. The screening may be done either because the compound is designed to have a pharmacological effect on liver cells, or because a compound designed to have effects elsewhere may have unintended hepatic side effects. Two or more drugs can be tested in combination (by combining with the cells either simultaneously or sequentially), to detect possible drug-drug interaction effects.

In some applications, compounds are screened initially for potential hepatotoxicity (Castell 1997). Cytotoxicity can be determined in the first instance by the effect on cell viability, survival, morphology, and leakage of enzymes into the culture medium. More detailed analysis is conducted to determine whether compounds affect cell function (such as gluconeogenesis, ureogenesis, and plasma protein synthesis) without causing toxicity. Lactate dehydrogenase (LDH) is a good marker because the hepatic isoenzyme (type V) is stable in culture conditions, allowing reproducible measurements in culture supernatants after 12-24 h incubation. Leakage of enzymes such as mitochondrial glutamate oxaloacetate transaminase and glutamate pyruvate transaminase can also be used (Gomez-Lechon 1996) describe a microassay for measuring glycogen, which can be applied to measure the effect of pharmaceutical compounds on hepatocyte gluconeogenesis.

Other current methods to evaluate hepatotoxicity include determination of the synthesis and secretion of albumin, cholesterol, and lipoproteins; transport of conjugated bile acids and bilirubin; ureagenesis; cytochrome p450 levels and activities; glutathione levels; release of alpha-glutathione s-transferase; ATP, ADP, and AMP metabolism; intracellular K⁺ and Ca²⁺ concentrations; the release of nuclear matrix proteins or oligonucleosomes; and induction of apoptosis (indicated by cell rounding, condensation of chromatin, and nuclear fragmentation). DNA synthesis can be measured as [³H]-thymidine or BrdU incorporation. Effects of a drug on DNA synthesis or structure can be determined by measuring DNA synthesis or repair. [³H]-thymidine or BrdU incorporation, especially at unscheduled times in the cell cycle, or above the level required for cell replication, is consistent with a drug effect: Unwanted effects can also include unusual rates of sister chromatid exchange, determined by metaphase spread (See generally, Vickers 1997).

The following examples provide further non-limiting illustrations of particular embodiments of the invention.

### EXAMPLE 1

### Hepatocyte Cell Cycle Progression is Substrate-Dependent

Regulation of ERK Activity by Collagen Structure

Primary rat hepatocytes cultured on a thin, monomeric films of type I collagen (Vitrogen™) spread and proceeded through one round of the cell cycle in the presence of epidermal growth factor (EGF) in defined, serum-free medium. In contrast, hepatocytes cultured on denatured collagen (Vitrogen) substrate maintained round morphology and did not proliferate. Cyclin D1 mRNA, protein, and associated kinase activity that are typically associated with an active cell cycle were absent in these cells. However, the level of other cell cycle-related molecules, e.g., p27, cyclin E, did not differ on the two substrates (Hansen 1999). These data indicate that signal transduction pathways upstream of cyclin D1 are inhibited on collagen gel, and the lack of cyclin D1 is a critical step in growth inhibition on this substrate.

Studies were initiated to identify potential differences in ECM-mediated signaling pathways induced by collagen film and gel. One pathway known to be initiated by both growth factor stimulation and adhesion is the mitogen-activated protein (MAP) kinase pathway. While different MAP kinase pathways have been identified, the one most commonly associated with both growth factor and ECM-induced activation was the p42/44 extracellular-regulated kinase (ERK 1 and 2) (Chen 1994, Morino 1995, Zhu 1995). Once activated by upstream kinases (i.e., MEKs, MKKs), ERKs are responsible for phosphorylating numerous proteins, including transcription factors.

To determine if ERK activation occurs on both collagen substrates (film and gel), phosphorylation of ERKs was measured using Western blot analysis and antibodies against the phosphorylated form of ERK 1/2 (New England Biolabs). ERKs are phosphorylated in freshly isolated hepatocytes compared to liver tissue, demonstrating that collagenase perfusion alone induces initial ERK phosphorylation. Once plated onto collagen film, ERK phosphorylation persisted and increased slightly over the first 2 hours in culture, then gradually diminished out to 24 hours. A second peak of phosphorylation was observed by 48 hours, just prior to entry into S phase. Both peaks of phosphorylation correlated with increased ERK kinase activity as determined by in vitro kinase assays. In contrast, the initial phosphorylation subsides more rapidly in cells on collagen gel and the second peak of ERK phosphorylation was absent. Kinase activity was also greatly reduced on collagen gel. The second peak of ERK activity, but not the first, appeared necessary for S phase, because inhibition of the upstream kinase MEK1 during late G₁ using U0126, a chemical inhibitor of MEK1, inhibits DNA synthesis, while inhibition during early G₁ does not.

Thus, ERK activation in late-G1 is inhibited by substrate conformation. This is the first demonstration of substrate-dependent late G1 ERK activity.

Regulation of Ras/RAF/MEK and EGF Receptor by Collagen Structure

Additional studies indicate that constitutively active (CA) forms of the genes expressing upstream activators of ERK (Ras, Raf, and MEK) were inactive on collagen gel. Over-expression of these ERK activators using adenoviral vectors led to restoration of progression through G1 and into S phase (DNA synthesis) in hepatocytes on collagen gel. These data demonstrate that cell cycle inhibition on collagen gel likely lies upstream of Ras, because active Ras can restore all downstream activities measured.

Activation of Ras requires signaling from growth factor receptors. Upon binding of epidermal growth factor (EGF), the EGF receptor (EGFr) transmits intracellular signals by autophosphorylation on tyrosine residues. Lysates from hepatocytes cultured on film or gel from 0.5 to 4 hours were immunoprecipitated with anti-phosphotyrosine (pTyr) antibody, then examined by Western blot using anti-EGFr antibody to determine if EGFr phosphorylation had occurred. Extensive EGFr phosphorylation was observed on both film and gel substrates compared to freshly isolated cells. Curiously, a similar analysis of lysates from cells cultured for 9 hours demonstrated persistent EGFr phosphorylation in cells cultured on film. However, such EGFr phosphorylation disappeared in cells cultured for longer times on gel. These data are the first indication that growth factor receptor phosphorylation is, in the long-term, substrate-dependent. Moreover, in light of the biphasic ERK response, such prolonged or biphasic EGFr phosphorylation may be required for a second peak of ERK activity.

Gel Inhibition of Cell Cycle Progression

To determine if inhibitory signals were generated by cell interaction with collagen gel, a study was designed in which hepatocytes were initially cultured on collagen film for 16 hours, thus inducing cell cycle progression, followed by addition of a room temperature, liquid Vitrogen solution on top of the film-plated cells. Because collagen polymerizes at 37° C., the added collagen forms a gel on top of the cells. Previous studies have defined such “collagen sandwich” culture conditions, in which hepatocytes remained viable and highly differentiated (Dunn 1989, Dunn 1992). However, no study had yet tested the effect of collagen gel overlay on cell cycle events already in progress.

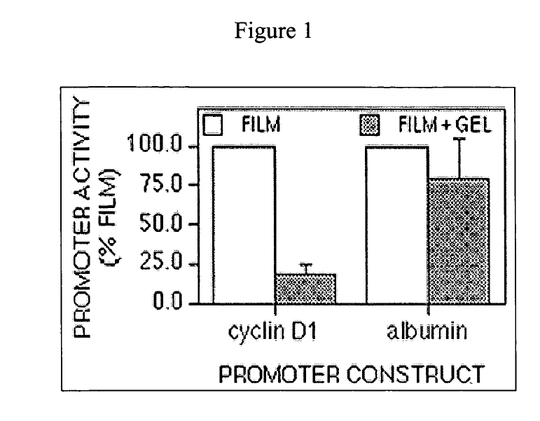

Cyclin D1 promoter activity was assessed 72 hours after initial plating, and DNA synthesis was measured at 60-72 hours after initial plating (44-56 hr after gel addition). Cell morphology initially remained unchanged, however, by two days after gel addition, hepatocytes appeared slightly less spread yet still clearly adherent to and partially spread on the collagen film. Cyclin D1 promoter activity was found to be greatly reduced compared to cells without the collagen overlay, while the activity of the albumin promoter remained essentially unchanged, demonstrating that diminished cyclin D1 promoter activity is not due to a general decrease in transcription (FIG. 1). Similarly, DNA synthesis was also diminished following collagen gel overlay. Thus, collagen gel overlay was able to inhibit cell cycle progression already in progress, suggesting that gel may induce specific signal(s) capable of inhibiting growth.

### EXAMPLE 2

### Protein Kinase A Inhibitors Stimulate Hepatocyte Cell Growth without Loss of Differentiation

Substrate-Dependent Signaling Pathways: Protein Kinase A

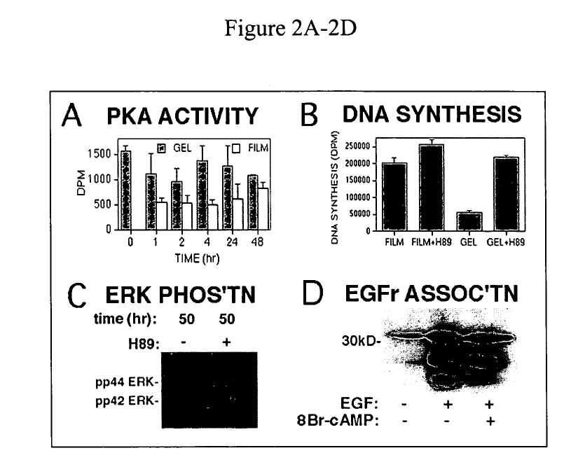

Protein kinase A (PKA) it is thought to inhibit several cell cycle signaling components and to be induced upon loss of adhesion (Howe 2000). Experiments were performed to address the possibility that protein kinase A acted as a growth inhibitor present or induced by collagen gel. The first step to determine if PKA was involved in growth inhibition on collagen gel was to measure PKA activity in hepatocytes cultured on the different substrates. PKA activity was determined using a modification of a published method (Day 1989). Data presented in FIG. 2A represents the mean±SD of PKA activity for duplicate samples of hepatocytes cultured on the different substrates after subtracting background (determined by running the assay in the absence of substrate). Virtually all of the [³²P] measured is attributable to PKA activity, because the protein kinase inhibitory peptide, PKI, specifically inhibits [³²P] incorporation. Moreover, throughout the 48 hr culture period, PKA activity remained higher in cells cultured on collagen gel than on film. Thus, PKA may play a role in inhibition of signaling events and cell cycle progression when cells are cultured on gel.

Once it was confirmed that PKA activity was higher in cells cultured on gel, PKA activity was modified to assess its role in cell cycle progression. H89, a specific inhibitor of PKA activity, was added to hepatocyte cultures on gel or film, and DNA synthesis and ERK phosphorylation were assessed. H89 significantly increased DNA synthesis on collagen gel to the level observed on film, while having little effect on film-plated cells (FIG. 2B). ERK phosphorylation was also significantly enhanced (FIG. 2C). In addition, EGFr phosphorylation was measured 9 hours after plating in the presence of H89 in hepatocytes cultured on collagen gel. While EGFr phosphorylation is decreased in hepatocytes cultured on gel by 9 hours, the presence of H89 led to maintenance of EGFr phosphorylation (FIG. 2D), suggesting that after the first four hours, when EGFr is phosphorylated normally, PKA down-regulates its phosphorylation and likely its activity during the later stages of G1. The fact that inhibition of PKA is sufficient to allow normal G1-S phase events to occur on collagen gel, indicates that adhesion to collagen gel can provide sufficient growth stimulatory signals, but that these are inhibited by simultaneous PKA activation.

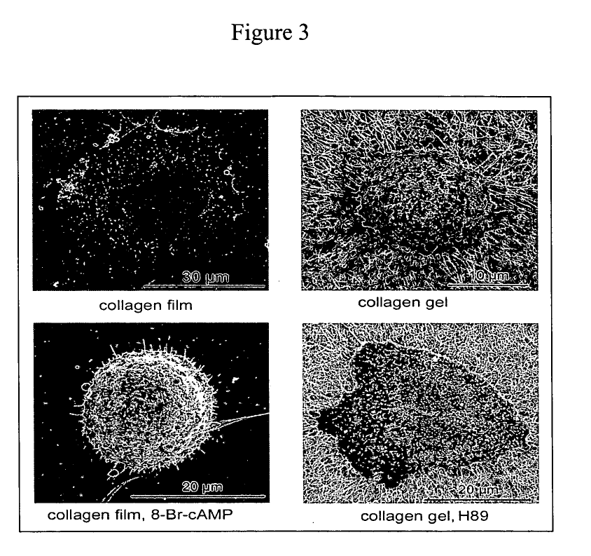

PKA inhibition also had surprising effects on cell morphology. It has been proposed that malleable substrates such as collagen gel are unable to promote cell spreading because it lacks sufficient mechanical strength to resist contractile forces applied by the cell to its adhesion sites. However, hepatocytes cultured on gel with H89 demonstrate significant spreading, while addition the PKA agonist, 8-Br-cAMP, significantly inhibits cell spreading on collagen film (FIG. 3). This result refutes the rigidity explanation for lack of spreading, and suggests, instead, that cell spreading on gel is due in large part to elevated PKA activity and a resulting inhibition of cytoskeletal rearrangements required for spreading.

It is clear from the results described above that hepatocyte progression through S phase of the cell cycle is promoted on collagen gel by inhibition of PKA. What is of additional interest is the ability of PKA inhibition to promote progression fully through the cell cycle, leading to proliferation and an increase in cell number. This ability would not only be of interest in terms of understanding basic regulatory mechanisms of substrate-dependent growth control, but it also may provide a mechanism by which hepatocytes could be propagated in vitro on a substrate (i.e., collagen gel) that maintains both viability and differentiated function.

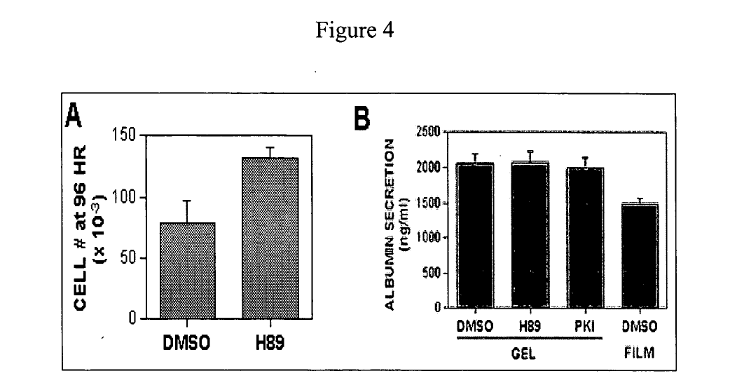

Cell counting experiments were performed to determine the number of hepatocytes 96 hours after plating on collagen gel in the presence or absence of H89. A 67% increase in cell number in the presence of H89 was observed compared to DMSO controls (FIG. 4A). While growth on collagen films was also high, hepatocytes quickly become de-differentiated on collagen films. Coincident with the higher cell number in cells cultured with H89 is an apparent increase in small hepatocyte-like cells in culture that may be newly divided daughter cells.

While the ability to increase hepatocyte number is important, other culture conditions, such as a collagen film, are able to produce a similar result. A true advance in this field, though, was the discovery of conditions that allowed for hepatocyte proliferation without a loss of differentiated function.

To determine if PKA inhibition had a detrimental effect on the differentiated function observed on collagen gel, albumin secretion was measured. Albumin is a protein synthesized and secreted by hepatocytes and this assay is routinely used as a marker of differentiated function. As shown in FIG. 4B, the level of albumin secretion after 48 hours in culture on collagen gel in the presence of H89 was not reduced compared to the DMSO controls. Similar results were obtained when PKA was inhibited using an adenoviral vector expressing the PKA inhibitory peptide, PKI. Urea production is also maintained on collagen gel in the presence of PKA inhibition (FIG. 4C). These data indicate that PKA inhibition is effective to promote hepatocyte proliferation in vitro without a significant loss of hepatocyte-specific function.

### EXAMPLE 3

### Identifying Substrates for Differentiating Stem Cells into Hepatocytes

The ECM composition with which developing cells contact varies at different stages of differentiation. During early liver development, hepatic endoderm cells in the liver bud migrate away from the cardiac tissue into the mesenchymal tissue of the septum transversum. This brings the cells into contact with a different ECM composition. The expression of liver-specific genes is coincident with this mesenchymal interaction, suggesting that the ECM composition plays a major role in directing differentiation into hepatocytes (Cascio 1991).

Stem cell differentiation is also highly influenced by the substrate to which the stem cells adhere. Studies have been performed on multipotent adult progenitor cells (MAPCs) to observe differentiation into hepatocyte-like cells on different extracellular matrices (ECMs), including fibronectin, type I collagen, and Matrigel™. In these studies, Matrigel™ generated a higher proportion of epithelioid cells (alb-,CK18-,HNF3β+) (61.4%), while fibronectin induced a slightly lower level (53.1%) and collagen produced virtually no epithelioid cells (Schwartz 2002). However, these MAPC differentiation studies compared Matrigel™ only to dishes coated with fibronectin or type I collagen, which yields a rigid film rather than a malleable gel. Studies presented here and elsewhere indicate that a variety of cell types respond differently to type I collagen when it is presented as a polymerized gel compared to a thin coating, or film (Hansen 1999).

Many stem cells, including the multipotent adult progenitor cells used in the present studies, differentiate more readily on gel-like substrates such as Matrigel™, suggesting that not only composition, but the mechanical nature of the substrate is instructive for differentiation. While Matrigel™ is a popular choice of substrate for induction of differentiation, several studies also indicate that type I collagen gel can be similarly inductive of differentiation when presented as a polymerized gel rather than a thin coating. Matrigel™ is a complex substrate derived from a sarcoma tumor cell line. It contains numerous cytokines and growth factors, as well as extracellular matrix components.

The “collagen gel sandwich”, or gel overlay condition promotes differentiated function in adult hepatocytes (Dunn 1989, Dunn 1992). The inventors found that this differentiated response was accompanied by a specific inhibition of the cell cycle even when hepatocytes are actively progressing through the cell cycle (FIG. 1).

While it is clearly advantageous to promote hepatic differentiation of multipotent adult progenitor cells, the development of a differentiation phenotype may be associated with diminished self-renewing capability as cells become more adult-like. Depending on the final application, it may be beneficial to keep multipotent adult progenitor cells in a less-differentiated form in order to optimize their proliferation to generate large cell numbers, then quickly induce differentiation once sufficient cell number is obtained. This is possible by propagating cells on fibronectin for several population doublings, followed by collagen overlay to induce differentiation. The ability to induce differentiation by simply applying an overlay to adherent cells rather than detaching and replating cells onto a different substrate is of great benefit in large scale practice.

(a) Comparison of the Following Substrates for their Ability to Induce MAPC Differentiation into Hepatocyte-Like Cells: Matrigel™, Fibronectin, Type I Collagen Film, and Type I Collagen Gel.

Multipotent adult progenitor cells are obtained and cultured on Fn for 50 population doublings as previously described (Reyes 2001). Multipotent adult progenitor cells have been isolated from mouse, rat, and human, and each species is able to differentiate into hepatocyte-like cells (Schwartz 2002).

Once multipotent adult progenitor cells achieve 50 population doublings, hepatocytes are initially plated in multipotent adult progenitor cell expansion medium (see below) onto four substrates for initial comparison: Matrigel™, fibronectin (film coating), type I collagen film, and type I collagen gel. The same attachment substrates with type I collagen gel overlay added 6 hours after plating are tested. Cells are initially plated in expansion medium for 12 hours, followed by a change to hepatocyte-differentiation medium as previously described (see below; (Schwartz 2002)). Following 28 day cultures, markers of hepatocyte differentiation are examined, including alb, CK18, and HNF3β. The percent positive cells for all these markers are quantified. Furthermore, additional stem cell and hematopoietic markers are examined to indicate lineage, as well as AFP and CK19 to indicate early hepatocyte progenitor phenotype. RT-PCR is used to measure gene expression patterns.

(b) Alter the Timing of Substrate Interaction and Add Collagen Overlay at Different Times to Determine Optimal Substrate Conditions for MAPC Differentiation into Hepatocyte-Like Cells.

To test whether different ECMs may provide optimal signals if provided at certain times within the differentiation pathway or in a given sequence, different ECM conditions are studied. The conditions are given below, with analysis taking place on day 28:
i. Fn 7 days; Matrigel™ 21 days
ii. Fn 7 days; Matrigel™ 21 days; collagen overlay at day 8
iii. Fn 28 days; collagen overlay at day 2
iv. Fn 28 days; collagen overlay at day 15
v. Matrigel™ 28 days, collagen overlay at day 2
vi. Matrigel™ 28 days, collagen overlay at day 15
vii. Type I collagen gel 28 days, collagen overlay at day 2
viii. Type I collagen gel 28 days, collagen overlay at day 15
   Analysis is as described above, i.e., immunohistochemistry of hepatocyte markers; RT-PCR.
   (c) Manipulate Specific Signaling Events, i.e., PKA, in the Differentiated Hepatocyte-Like MAPCs Once Further Differentiation and/or Proliferation is Induced.

MAPCs differentiate into hepatocyte-like cells within 14 days of culture on Matrigel in defined medium (Schwartz 2002). The effect of this differentiation on proliferative capacity has not been examined.

Intracellular signaling events induced in MAPCs during the undifferentiated state and on the different substrates in hepatocyte-promoting medium are defined in a similar manner as in adult hepatocytes. The expression of G1 phase cell cycle regulatory proteins, including ERKs, cyclin D1, cyclin E, and p27, is examined using Western blot analysis. Phospho-specific antibodies for ERK are utilized. Because progression through M phase is of interest as well, cyclin B is added to the panel. PKA activity is assessed using the in vitro kinase assay (Day 1989). The substrates tested include Matrigel, Matrigel+ collagen gel overlay, Fn, Fn+ collagen gel overlay, and type I collagen gel.

In addition to the cell cycle markers, differentiation-specific markers are examined. Such markers include functional markers such as albumin secretion, urea production, and PROD assays. In addition, differentiation-specific gene expression is examined using QRT-PCR. Of particular interested is the expression of liver-specific transcription factors, e.g., HNFs, C/EBPα and β, as well as cytochrome P450 enzymes. While gene expression does not necessarily indicate function, the pattern of expression of these genes is a key to unveiling regulatory mechanisms governing the switch between a proliferative and differentiated phenotype in both undifferentiated MAPCs and hepatocyte-like cells.

Methods:

MAPC Generation: BM is obtained from Sprague Dawley rats, and MAPCs will be isolated and cultured as previously described for human MAPCs (Reyes 2001). Briefly, MAPCs are plated at 5-10×10³ per well of a Fibronectin-coated 96-well plate in expansion medium (60% low-glucose DMEM (Gibco BRL), 40% MCDB-201 (Sigma), 1× insulin transferrin selenium, 1× linoleic acid bovine serum albumin, 10⁻⁹M dexamethasone (Sigma), 10⁻⁴M ascorbic acid 2-phosphate (Sigma), 100 U penicillin, 1000 U streptomycin, 2% FCS (Hyclone Labs, Logan, Utah), 10 ng/ml EGF, 10 ng/ml LIF (Chemicon International, Temecula, Calif.) and 10 ng/ml PDGF-BB (R&D Systems, Minneapolis, Minn.). Once adherent cells are more than 50% confluent, they are detached with 0.25% trypsin-EDTA (Sigma) and replated at a 1:4 dilution under the same culture conditions.

Substrate Preparation: COLLAGEN FILM: Non-adhesive plastic Petri dishes are coated overnight at 4° C. with type I collagen (Vitrogen® collagen, Cohesion Corp, Palo, Alto, Calif.) diluted in carbonate buffer (15 mM sodium carbonate, 35 mM sodium bicarbonate, pH 9.4) at 1 μg/cm². COLLAGEN GEL: Vitrogen® collagen is diluted 4 parts Vitrogen® collagen: 1 part 5× Williams Medium E (Gibco), and 0.02 parts 1N NaOH added to a Petri dish at 1 ml/100 cm², and incubated at 37° C. for 1 hr. FIBRONECTIN: The coating procedure is identical to that used for collagen film. 1 μg/cm² fibronectin in carbonate buffer (see above) will be added to plates at 4° C. overnight. MATRIGEL: 1% Matrigel™ solution (Becton-Dickinson and Co., Franklin Lakes, N.J.) will be added to culture dishes. All plates are rinsed twice in PBS, followed by blocking 30 minutes in William's E with 1% bovine serum albumin.

MAPC Differentiation: After 50 population doublings, MAPCs are trypsinized and replated onto the appropriate substrates. After 8-12 hours, media is removed, cells washed with PBS, and fresh medium is added to promote hepatocyte differentiation (expansion medium supplemented with 10 ng/ml FGF-4 and 20 ng/ml HGF).

Immunohistochemistry: MAPC staining for differentiation-specific markers takes place as previously described (Schwartz 2002).

Total RNA isolation and quantitative RT-PCR: Methods for RT-PCR and primer sequences are as previously described (Schwartz 2002).

PKA In vitro Assay: As previously described (Day 1989), cells are lysed, sonicated, centrifuged, and total protein determined. Equal protein is added to reaction mixture along with substrate (Kemptide, a synthetic peptide containing the consensus PKA phosphorylation sequence), and 5 μCi γ-³²P ATP. After 30° C. incubation for 10 minutes, 25 μl is spotted onto Whatman P81 paper, washed extensively in 75 mM phosphoric acid, dried, and quantified by scintillation.

Immunoprecipitation, SDS-PAGE, Western blot: Immunoprecipitation (IP) of EGFr-associated proteins will be performed (Saso 1997, Vacca 2000). Detection will consist of the ECL Plus chemiluminescence system (Amersham) and phosphorimager analysis (Molecular Dynamics) for imaging and quantification.

DNA Synthesis: DNA synthesis will be measured using [³H]-thymidine incorporation in substrate-coated 96-well plates, as previously described (Hansen 1999). DPMs will be normalized to cell number assessed in parallel plates using the CyQuant Assay (Molecular Probes).

Proliferation: Hepatocytes cultured on 35 mm dishes will be removed from plates using a combination of trypsin, collagenase, and DNAse at various times after plating. Cells are counted using a Coulter counter and hemocytometer. A cell count at 24 hour is also determined to assess the initial number of viable, attached cells. Plates are run in triplicate.

### EXAMPLE 4

### Optimizing Culture Conditions

PKA affects many cellular processes, including many events at different points in the cell cycle. Published studies also demonstrate that PKA regulation plays an important role in hepatocyte proliferation during liver regeneration (Ekanger 1989, Roth 1990). The data provided herein indicates that inhibiting elevated PKA on collagen gel allows progression through S phase of the cell cycle. Data also suggest that PKA inhibition promotes full cell cycle progression, as seen by the increased cell number on gel 96 hr after H89 addition (FIG. 4). PKA, however, also participates in the G2/M phase transition. PKA activity is typically low as cells approach M phase, and activation of PKA inhibits entry into mitosis (Grieco 1996). In late M phase, PKA activity goes up (Grieco 1996), and it is recruited into chromatin (Collas 1999), where it is postulated to be dynamically involved in chromatin remodeling (Landsverk 2001). Blocking this late M phase PKA activation prevents the transition into interphase

A 67% increase in primary adult hepatocyte numbers was observed following PKA inhibition (FIG. 4). Tests are also performed to promote repeated cell cycles to greatly expand the cell population. A certain basal level of PKA activity is required for cell viability and maintenance of basic housekeeping functions. Thus, in some circumstances one may not desire to maintain constant PKA inhibition. In other circumstances, one may choose to expose cells to PKA inhibitor at specific intervals to provide sufficient PKA inhibition to allow cell cycle progression without fully eliminating PKA activity.

Regulation of PKA activity by substrate adhesion has been demonstrated both in our studies with hepatocytes as well as in other cell types. The role of cell-cell interaction in regulating PKA activity is less clear. Cell-cell contact certainly has a well-documented inhibitory effect on cell cycle progression, as demonstrated by contact inhibition in all virtually non-transformed cells. Cell-cell contact also appears to facilitate differentiated function. Hepatocyte spheroids, or self-assembled aggregates of hepatocytes, possess possibly the highest degree of differentiated function documented in vitro.

(a) Vary the Timing of PKA Inhibition to Target Specific Points in the Cell Cycle for Optimal Proliferation.

To this point, experiments consisted of PKA inhibition by H89 for 96 hr cultures and counts at either 96 or 120 hours. However, reports suggest that during normal cell cycle progression of fibroblasts, PKA activity is low as cells enter M phase, but becomes elevated at the end of M phase (Grieco 1996). This PKA elevation appears to be necessary for transition into interphase. The timing of PKA inhibition is varied in order to establish an optimal treatment for induction of proliferation. The initial sequences to be tested are:

| | | APPROXIMATE |
|---|---|---|
| | | CORRESPONDING CYCLE |
| TREATMENT TIME | | PHASE |
| i. | H89 0-120 hr, cell counts at 120 hr | inhibition throughout cell cycle |
| | | and into interphase |
| ii. | H89 0-96 hr, cell counts at 120 hr | inhibition through M phase |
| iii. | H89 0-80 hr, cell counts at 120 hr | inhibition through S phase |
| iv. | H89 0-48, 80-96 hr, | inhibition through G1 and |
| | cell counts at 120 hr | G2/M phases |
| v. | H89 0-48 hr, cell counts at 120 hr | inhibition through G1 phase |

Cells are plated in 35 mm dishes in triplicates for each condition. Cell counts are determined at 120 hours, assessed by both hemocytometer and Coulter counter for each plate. In addition, cell lysates are obtained at 48, 72, 84, 96, and 120 hours for Western blot analysis of cyclin D1 (late G1) and cyclin B (late G2/M) expression to assess progression through the different cell cycle stages. In addition, because primary hepatocytes can be bi-nucleated, nuclear counts are assessed by DAPI staining and quantification using digital analysis. Both nuclear and cellular morphology is assessed by photomicrography at 72, 96, and 120 hours. Nuclear morphology assessment is a key in determining the proper progression through M phase. The presence of mitotic figures is assessed as well as determining the proportion of resulting hepatocytes with mono-, bi-,and multi-nuclear morphology.

PKA activity is assessed throughout the 120 hour culture interval, with and without the above inhibition schedules. Activity is first assessed in the absence of any inhibition to determine the precise levels of PKA activity throughout normal proliferation on collagen film, as well as over the same time period on collagen gel. Once this is determined, we will assess PKA activity in a subset of the above experiments, e.g.,12 hrs following removal of H89, to assure that H89 removal will result in return of elevated PKA on gel.

PKA dosage is also tested. A range of PKA dose, namely, 0.1, 0.5, 1, 3, and 5 μM, is tested for its ability to promote increased cell counts at 120 hours following the H89 treatment to provide optimum proliferative response. Triplicate cell counts are determined, as well as simple trypan blue stain to assess viability.

(b) Determination of the Ability of PKA Inhibition at Intervals to Induce Repeated Proliferative Cycles.

The ability of hepatocytes to undergo repeated rounds of proliferation in vitro to greatly expand cell number greatly enhances the ability to utilize primary hepatocytes in cell based therapies. PKA inhibitor is administered at intervals subsequent to the first round of proliferation. Cultures are continued for various times without PKA inhibition to allow for interphase, then PKA inhibitor is applied again and additional proliferation is assessed. The experiments are carried out using the following schedule:

Treatment Time

i. H89 0-96 hr, no H89 12 hr, H89 another 96 hr (108-204 hr), cells counts at 228 hr
ii. H89 0-96 hr, no H89 24 hr, H89 another 96 hr (120-216 hr), cells counts at 240 hr
iii. H89 0-192 hr (2 putative cell cycle rounds), no H89 24 hr, cell counts at 216 hr
iv. H89 0-96 hr, no H89 48 hr, H89 another 96 hr (144-240 hr), cells counts at 264 hr
v. H89 0-96 hr, no H89 72 hr, H89 another 96 hr (168-264 hr), cells counts at 288 hr
vi. H89 0-96 hr, no H89 96 hr, H89 another 96 hr (192-288 hr), cells counts at 312 hr

Cell counts are performed at the end of the “no H89” period (except in iii) to assess cell count prior to the second H89 treatment, and again at the end of the experiment as indicated above. A cell count is also performed at 24 hours to verify the number of attached cells prior to proliferation. Triplicate plates are run for each cell count under each condition (9 plates total for each condition). Additional endpoints to be examined include viability by trypan blue staining and TUNEL staining to determine the level of apoptosis, if any.

(c) Determine the Effects of Cell Density on the Ability of PKA Inhibition to Promote Cell Cycle Progression.

Extensive cell-cell contact in vitro, or cell confluence, is inhibitory for cell growth. There may be a minimal number of cells required for a proliferative response in the presence of PKA inhibitors, as there is in a number of cell cultures. This is systematically tested in this experiment in order to establish the ideal cell density needed to achieve optimum proliferation. It is also necessary to determine if cell number increases, at what density and time period would hepatocytes need to be “split” to maintain viability and proliferative capacity.

Hepatocytes attain significant cell-cell contact to inhibit DNA synthesis between 25-50×10³ cells/cm². Thus cell densities tested range from approximately 5×10³ to 50×10³/cm². The density used in many of the studies presented in Preliminary Studies was approximately 10-12×10³/cm², which is intermediate among the densities to be tested.

(d) Test Long Term Effects (e.g., Out to Two Months) of PKA Inhibition on Hepatocyte Viability and Differentiated Function.

Once a schedule of PKA inhibition is determined that promotes optimal propagation of hepatocytes with desired maintenance of differentiated function, the treatment's effect on long term viability and function is tested. Several methods exist to measure differentiated function. Those methods described in the previous Examples are used, specifically functional assays (albumin, urea, and PROD) and gene expression studies of differentiation-specific genes, focusing on liver-enriched transcription factors. Viability by trypan blue staining and assessment of apoptosis as measured by TUNEL assay (terminal deoxynucleotidyl transferase biotin-dUTP nick end labeling) is used as markers of viability and apoptosis, respectively. Cultures are maintained for two months after H89 treatment, if possible, with tests of viability and function every two weeks after an initial assessment every other day for the first 2 weeks.

Methods:

Hepatocyte Culture: Cells are obtained by collagenase perfusion of adult Lewis rat liver (Aiken 1990), followed by purification through a Percoll gradient (Sigma, St. Louis, Mo.). Hepatocytes will be plated (12,000 cells/cm²) in serum-free William's medium E with defined additives including EGF (10 ng/ml, Collaborative Research, Bedford, Mass.) and insulin (20 mU/ml, Sigma) (Hansen 1994).

TUNEL Assay: The TUNEL staining kit from Upstate Biologicals will be used for immunofluorescent assessment of apoptosis in hepatocyte cultures.

### EXAMPLE 5

### Developing Optimal Bioartificial Liver Devises

Propagation of hepatocytes in vitro on collagen gel by PKA inhibition represent a great advance in the ability to use hepatocytes for in vitro cultures, including tests of drug toxicity and metabolism. Additional uses for hepatocytes are also contemplated. Furthermore, due to the native three-dimensional nature of the in vivo liver tissue environment, it may be beneficial to culture hepatocytes either within the collagen gel, or on a film or gel with gel overlay, depending on the ultimate application. This may better mimic the in vivo environment, leading to improved differentiated and potentially proliferative phenotype.

One of the most important applications involving hepatocytes for clinical use is the bioartificial liver extracorporeal device for temporary liver support. While several different designs exist, each device could benefit from increasing hepatocyte cell mass, resulting in higher reactor function. The set-up procedure itself often leads to significant cell loss, and the ability to replace that lost cell mass by inducing one or more rounds of proliferation could greatly improve the final reactor function.

The loss of initial cell viability and function is particularly true in procedures involving cryopreservation or other long-term storage procedures. The need to provide reactors at distant sites requires such injurious procedures, with the cell population clearly experiencing detrimental effects. Apoptosis occurs during cryopreservation, yet may be reversed by incubation with caspase inhibitors during the cryopreservation process (Yagi 2001). An additional boost by increasing cell number or further promoting survival during culture provides further benefit.

(a) Determination of the Ability of PKA Inhibition to Promote Hepatocyte Progression Through the Cell Cycle when Embedded Within Rather than Sitting on Top of a Collagen Gel.

Certain BAL designs, such as the Minnesota Bioartificial Liver device (Nyberg 1993, Nyberg 1993b), utilize hepatocytes embedded within a type I collagen gel inside the fibers of the hollow fiber reactor. The first step in determining effects of PKA inhibition on this reactor design is to test hepatocyte signaling and response to PKA inhibition when they are embedded within, rather than sitting on top of the collagen gel, which has been the condition in studies thus far. This is accomplished by simply mixing freshly isolated hepatocytes within a solution of type I collagen, pouring into a dish, and allowing to polymerize for several hours at 37° C. Once the gel has polymerized, medium will be changed to that containing H89 for the optimal schedule and dose determined above. DNA synthesis, cell number, mitotic nuclei, and cyclin D1 and B markers are initially assessed to determine cell cycle progression under these conditions.

(b) Place Hepatocytes in a Mini-Collagen-Gel Based Bioreactor and Test the Effects of PKA Inhibition (Both Prior to and During Reactor Incubation) on Cell Cycle Progression and Differentiated Function.

The ability of signal manipulation to enhance cell function by increasing cell mass either through enhanced viability or cell proliferation is assessed in a “mini-BAL,” or small scale hollow fiber reactor cartridge. Rat hepatocytes are mixed in a solution of type I collagen (Vitrogen) then infused into the fibers within the reactor, as previously described (Nyberg 1993a, Nyberg 1993b). Reactors are then placed at 37° C. for 18 hr to allow for both collagen gel polymerization followed by cell-dependent contraction of the collagen gel. This contraction creates a second compartment within the fibers through which serum-free defined William's E media will be perfused after the 18 hr contraction period. It is to this medium that H89 or other chemical mediators of interest are added to allow direct (or very closely so) interaction between medium and cells. The extra-fiber space represents the compartment through which ultimately the patient's blood is perfused. For these in vitro studies, a separate media stream is perfused through this space and samples collected for functional analyses. Endpoints include oxygen consumption as an indirect indicator of viability, albumin secretion, and urea production. All experiments are performed in triplicate. It is also possible to extrude the hepatocyte-containing collagen “noodles” from the reactor fibers, perform cell lysis, and analyze cell cycle protein expression. Four week cultures are generally assessed, with functional assessment every two days for the first two weeks, then weekly for the remaining two week.

(c) Examine the Ability of Hepatocytes Attached to Collagen-Coated Beads in Suspension to Proliferate With or Without PKA Inhibition.

Another BAL design involves attaching primary hepatocytes to collagen-coated (Cytodex-3) beads, then infusing this bead-cell suspension into the extra-fiber space of a hollow fiber reactor, with media perfusion through the intra-fiber space (Rozga 1993). Because the beads are larger than the cells, this represents a condition more comparable to collagen film than the gel cultures. These conditions are directly compared by comparing proliferative and functional endpoints of the bead-cell suspension to that of gel-embedded cultures. Endpoints include albumin secretion, urea production, and PROD assay, as well as [³H]thymidine uptake to measure DNA synthesis.

These types of cultures are amenable to cell lysis and analysis of gene expression and signaling pathways, so endpoints of analysis include cell cycle protein expression to determine proliferative potential in the presence or absence of PKA inhibition. These results lend great insight into the mechanisms by which application-specific culture conditions regulate the determination of a proliferative versus differentiated phenotype.

(d) Test the Effects of PKA Inhibition on Cell Cycle Progression of Pig and Human Hepatocytes.

Pig hepatocytes are commonly used in BAL models, so their response to different culture conditions and PKA manipulation are assessed. A subset of substrate conditions are chosen based on previous studies, including collagen film, collagen gel, Fn with collagen gel overlay, and Matrigel. Pig hepatocyte proliferation and differentiation in the absence or presence of PKA inhibition is assessed. Endpoints include those described above (cyclin D1, E, and B protein analysis, DNA synthesis, cell counts, nuclear morphology assessment, albumin secretion, urea production, and PROD activity).

Human hepatocytes are also tested under the same conditions. Human cells are the obvious choice for both in vivo cell transplantation applications as well as in vitro drug testing.

Methods:

BAL Set-Up: Hepatocytes are mixed with Vitrogen solution and infused into small bioreactors as previously described (Nyberg 1993a, Nyberg 1993b). Following 18 hr gel polymerization and contraction at 37° C. during which media is perfused through the extracapillary space, an additional media stream is established in the intralumenal space created by gel contraction.

Pig Hepatocyte Harvest: Primary pig hepatocytes are performed as previously described (Sielaff 1995).

Bead-Attached Suspension Cultures: Primary rat hepatocytes are isolated and incubated in suspension cultures in the presence of Cytodex-3 beads, as previously described (Rozga 1993).

Human Cell Culture: Cells are obtained through LTPADS as described above. Human hepatocytes require trypsinization from the provided plates, followed by viability and cell concentration assessment, and replating onto defined substrates.

### EXAMPLE 6

### Enhanced Hepatocyte Number by PKA Inhibition

Protocol for Cell Counts

Coating: 60 mm plates were coated with film or 5× gel, as described above, with 4 replicates per condition.

Cells: Cells were added at 10000 cells/cm² in William's Media E Complete, with 3.3 μM H89, or with equal volume of DMSO.

Culture: The cells were cultured as described above, changing media in all plates at 72 hrs, and again at 96 hrs (day 4). Media in “No H89 d4” samples is changed from H89-containing to DMSO-containing, but remains the same for “DMSO” and “H89” samples.

Harvest: Gel—media was removed from plates. Five mL collagenase (approx. 1 mg/ml) in PBS was added to plates and incubated for 20 minutes at 37° C. Three mL trypsin-EDTA was added directly to collagenase solution and incubated another 15 minutes at 37° C. Media was removed with a pipette and plate was washed several times with the media, before the media was collected into a 15 mL tube. Tubes were centrifuged at 1000 rpm for 10 minutes at 4° C. Supernatant was removed and pellets from each of the four replicates were resuspended in 1 mL fresh media. Tube was weighed for end volume and equalized to 1 ml later.

Film—Media was removed from plates. Trypsin-EDTA was added to dishes (3 ml/dish) and dishes were incubated at 37° C. for 15 minutes. Procedure was repeated.

Samples were snap frozen in ethanol at −70° C. before thawing and counting.

Counting: Hemocytometer—100 μL sample was added to 900 μL trypan blue in PBS (1:4 dilution). Cells were counted. Repeated procedure seven times for eight total counts per sample.

Coulter Counter—Added 250 μL sample to 20 mL PBS in small Coulter vessel. Counted with predetermined pre-sets, collecting 1 mL of 20 mL sample and recording result. Repeated procedure seven times for eight total counts per sample.

Discussion

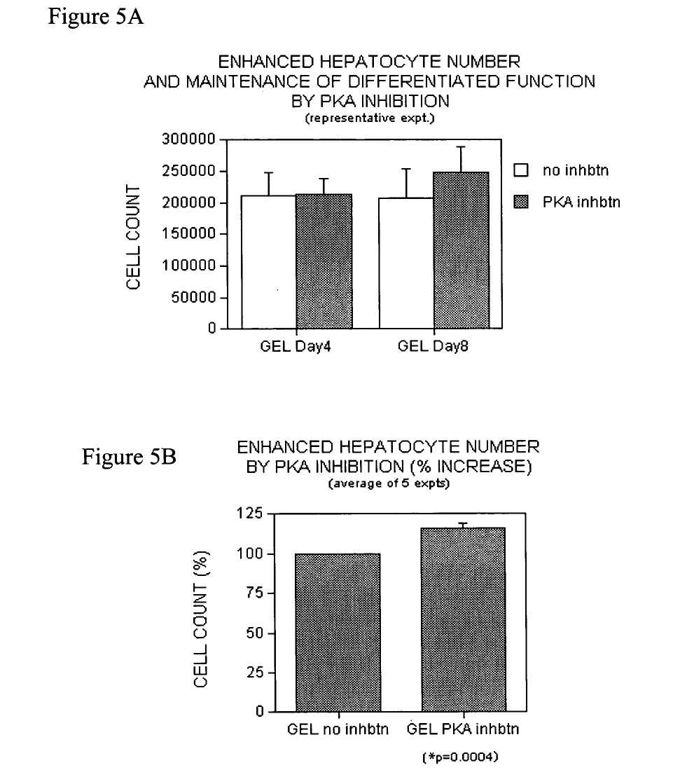

The inventors have now demonstrated an improvement in hepatocyte cell number after eight days in culture on type I collagen gel (also called “polymerized” or “fibrillar” collagen) following inhibition of Protein Kinase A (PKA) using H89. They obtained about 15-20% higher cell number (not just DNA synthesis, but actual cell counts) with PKA inhibition. The average of five experiments yielded an average 16% increase (16%±3.3%, p-value=0.0004). FIG. 5A shows one representative experiment, demonstrating no change in cell number at four days, and an increase at day eight. FIG. 5B is a graph that shows the average data from five experiments.

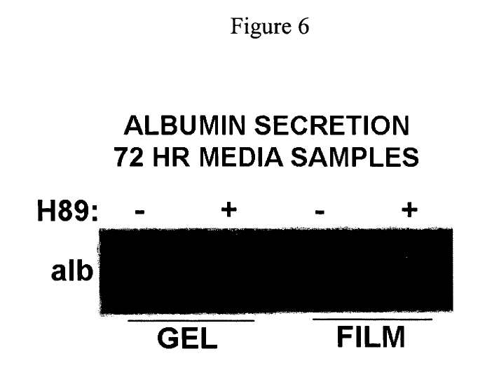

The observed enhanced cell number was accompanied by NO change in albumin secretion, which is a standard measure of differentiated function. FIG. 6 (not quantitated) shows a “dot blot” analysis of media samples from gel cultures with or without H89 after 72 hours. It also compares it to a thin film of type I collagen on which hepatocytes may proliferate but also lose differentiated function. Inhibition of PKA has little effect on DNA synthesis, cell counts, or albumin secretion in hepatocytes cultured on film.

It should be noted that the inventors also determined that a pulse of PKA inhibitor is preferable to a constant exposure to PKA. If the PKA inhibitor is left on for the entire culture period, there is no enhanced cell number, presumably due to the requirement of PKA in later stages of the cell cycle. Thus, the methodology differs slightly from our initial report in which we simply stated the inhibitor is added to the culture. In the present experiments, the inventors exposed the cells to the inhibitor on for the first 72 hours, removed it for the next 24 hours, and then added it back again.

It should also be noted that a typical hepatocyte cell cycle is about 4-5 days. Thus, after eight days (the end of most of experiments performed thus far), most cells were not be expected to have completed more than one full cell cycle. Thus, it is believed that the 16% increase in total cell numbers represents what can be achieved from one cell cycle. It is believed that continued intermittent inhibition of subsequent cell cycles will further increase the number of cells present.

All publications, patents and patent documents are incorporated by reference herein, as though individually incorporated by reference. The invention has been described with reference to various specific and preferred embodiments and techniques. However, it should be understood that many variations and modifications may be made while remaining within the scope of the invention.

Literature Cited

U.S. Pat. No. 4,491,632
U.S. Pat. No. 4,472,500
U.S. Pat. No. 4,444,887
U.S. Pat. No. 5,445,934
U.S. Pat. No. 5,474,895
U.S. Pat. No. 5,514,785
U.S. Pat. No. 5,030,015
U.S. Pat. No. 5,290,684
U.S. Pat. No. 5,624,840
U.S. Pat. No. 5,837,234
U.S. Pat. No. 5,853,717
U.S. Pat. No. 5,935,849
U.S. Pat. No. 6,294,380
U.S. Pat. No. 5,866,420
U.S. Pat. No. 5,605,835
U.S. Pat. No. 5,595,909
U.S. Pat. No. 4,853,324
U.S. Pat. No. 4,675,002
International Patent Application WO 01/11011
International Patent Application WO 94/13804
International Patent Application WO 92/01047
International Patent Application WO 90/02809
1. Schwartz, R. E. and e. al. (2002) Multipotent adult progenitor cells from bone marrow differentiate into functional hepatocyte-like cells. J. Clin. Invest. 109: 1291-302.
2. Jiang, Y., B. N. Jahagirdar, R. L. Reinhardt, R. E. Schwartz, C. D. Keene, X. R. Ortiz-Gonzalez, M. Reyes, T. Lenvik, T. Lund, M. Blackstad, J. Du, S. Aldrich, A. Lisberg, W. C. Low, D. A. Largaespada and C. M. Verfaillie (2002) Pluripotency of mesenchymal stem cells derived from adult marrow. Nature 418: 41-8.
3. Block, G. D., J. Locker, W. C. Bowen, B. E. Petersen, S. Katyal, S. C. Strom, T. Riley, T. A. Howard and G. K. Michalopoulos (1996) Population expansion, clonal growth, and specific differentiation patterns in primary cultures of hepatocytes induced by HGF/SF, EGF and TGF alpha in a chemically defined (HGM) medium. J. Cell Biol. 132: 1133-49.
4. Nyberg, S. L., R. A. Shatford, M. V. Peshwa, J. G. White, F. B. Cerra and W.-S. Hu (1993) Evaluation of a hepatocyte entrapment hollow fiber bioreactor: a potential bioartificial liver. Biotech. Bioeng. 41:194-203.
5. Rozga, J., F. Williams, M.-S. Ro, D. F. Enuzil, T. D. Giorgio, G. Backfisch, A. D. Moscioni, H. R. and A. A. Demetriou (1993) Development of a bioartificial liver: Properties and function of a hollow-fiber module inoculated with liver cells. Hepatol. 17: 258-65.
6. Matas, A. J., D. E. R. Sutherland, M. W. Steffes, S. M. Mauer, A. Lowe, R. L. Simmons and J. S. Najarian (1976) Hepatocellular transplantation for metabolic deficiencies: Decrease of plasma bilirubin in Gunn rats. Science 192: 892-4.
7. Vacanti, J. P., M. A. Morse, W. M. Saltzman, A. J. Domb, A. Perez-Atayde and R. Langer (1988) Selective cell transplantation using bioabsorbable artificial polymers as matrices. J. Ped. Surg. 23: 3-9.
8. Hansen, L. K. and J. P. Vacanti (1992) Hepatocyte transplantation using artificial biodegradable polymers. Current Controversies in Biliary Atresia (Eds: Hoffman, M.) Austin, R. G. Landes Publishing Company. 96-106.
9. Nyberg, S. L., R. P. Remmel, H. J. Mann, M. V. Peshwa, W.-S. Hu and F. B. Cerra (1994) Primary hepatocytes outperform HepG2 cells as the source of biotransformation functions in a bioartificial liver. Ann. Surg. 220: 59-67.
10. Albrecht, J. H. and L. K. Hansen (1999) Cyclin D1 promotes mitogen-independent cell cycle progression in hepatocytes. Cell Growth and Diff. 10: 397-404.
11. Hansen, L. K. and J. H. Albrecht (1999) Regulation of hepatocyte cell cycle progression by type I collagen matrix: Role of cyclin D1. J Cell Sci. 112: 2971-81.
12. Nelsen, C. J., D. G. Rickheim, N. A. Timchenko, M. W. Stanley and J. H. Albrecht (2001) Transient expression of cyclin D1 is sufficient to promote hepatocyte replication and liver growth in vivo. Cancer Res. 61: 8564-8.
13. Nelson, C. J., L. K. Hansen, D. G. Rickheim, C. Chen, M. W. Stanley, W. Krek and J. H. Albrecht (2001) Induction of hepatocyte proliferation and liver hyperplasia by the targeted expression of cyclin E and skp2. Oncogene 20: 1825-31.
14. Petersen, B. E., W. C. Bowen, K. D. Patrene, W. M. Mars, A. K. Sullivan, N. Murase, S. S. Boggs, J. S. Greenberger and J. P. Goff (1999) Bone marrow as a potential source of hepatic oval cells. Science 284: 1168-70.
15. Sell, S. (2001) Heterogeneity and plasticity of hepatocyte lineage cells. Hepatology 33: 738-50.
16. Suzuki, A., Y. W. Zheng, S. Kaneko, M. Onodera, K. Fukao, H. Nakauchi and H. Taniguchi (2002) Clonal identification and characterization of self-renewing pluripotent stem cells in the developing liver. J. Cell Biol. 156: 173-84.
17. Suzuki, A., Y. Zheng, R. Kondo, M. Kusakabe, Y. Takada, K. Fukao, H. Nakauchi and H. Taniguchi (2000) Flow-cytometric separation and enrichment of hepatic progenitor cells in the developing mouse liver. Hepatology 32: 1230-9.
18. Paolucci, F. and e. al. (1990) Immunohistochemical identification of proliferating cells following dimethylnitrosamine-induced liver injury. Liver Growth and Repair 10: 278-81.
19. Yin, L. D. L., Z. Ilic, S. Sell and R. Articles (2002) Proliferation and differentiation of ductular progenitor cells and littoral cells during the regeneration of the rat liver to CCL4/s-AAF injury. Histol. Histopathol. 17:
20. Yin, L., D. Lynch and S. Sell (1999) Participation of different cell types in the restitutive response of the rat liver to periportal injury induced by allyl alcohol. J. Hepatol. 31: 479-507.
21. Paku, S., J. Schnur, P. Nagy and S. S. Thorgeirsson (2001) Origin and structural evolution of the early proliferating oval cells in rat liver. Am. J. Pathol. 158: 1313-23.
22. Alison, M. and C. Sarraf (1998) Hepatic stem cells. J. Hepatol. 29: 678-83.
23. Tateno, C. and K. Yoshizato (1996) Growth and differentiation in culture of clonogenic hepatocytes that express both phenotypes of hepatocytes and biliary epithelial cells. Am. J. Pahtol. 149: 1593-605.
24. Gordon, G. J., G. M. Butz, J. W. Grisham and W. B. Coleman (2002) Isolation, short-term culture, and transplantation of small hepatocyte-like progenitor cells from retrorsine-exposed rats. Transplantation 73: 1236-43.
25. Doetschman, T. C., H. Eistetter, M. Katz, W. Schmidt and R. Kemler (1985) The in vitro development of blastocyst-derived embryonic stem cell lines: formation of visceral yolk sac, blood islands and myocardium. J. Embryol. Exp. Morphol. 87: 27-45.
26. Thompson, J. A. and e. al. (1998) Embryonic stem cell lines derived from human blastocysts. Science 282: 114.
27. Pittenger, M. F. and e. al. (1999) Multilineage potential of adult human mesenchymal stem cells. Science 284: 143-7.
28. Ferrari, G. and e. al. (1998) Muscle regeneration by bone marrow-derived myogenic progenitors. Science 279: 528-30.
29. Gussoni, e. and e. al. (1999) Dystrophin expression in the mdx mouse restored by stem cell transplantation. Nature 401: 390-4.
30. Orlic, D. and e. al. (2001) Bone marrow cells regenerate infarcted myocardium. Nature 410: 701-5.
31. Jackson, K. and e. al. (2001) Regeneration of ischemic cardiac muscle and vascular endothelium by adult stem cells. J. Clin. Invest. 107: 1395-402.
32. Lin, Y., D. J. Weisdorf, A. Solovey and R. P. Hebbel (2000) Origins of circulating endothelial cells and endothelial outgrowth from blood. J. Clin. Invest. 105: 71-7.
33. Lagasse, E. and e. al. (2000) Purified hematopoietic stem cells can differentiate into hepatocytes in vivo. Nat. Med. 6: 1229-34.
34. Theise, N. D. and e. al. (2000) Derivation of hepatocytes from bone marrow cells in mice after radiation-induced myeloablation. Hepatology 31: 235-40.
35. Reyes, M. and e. al. (2001) Purification and ex vivo expansion of postnatal human marrow progenitor cells. Blood 98: 2615-25.
36. Hansen, L. K., J. R. Friend, R. Remmel, F. B. Cerra and W.-S. Hu (1998) Development of a Bioartificial Liver Device. Methods in Molecular Medicine: Tissue Engineering Methods and Protocols (Eds: Morgan, J. R. and M. L. Yarmush) 18. Totowa, N.J., Humana Press Inc. 423-31.
37. Fassett, J. T., and L. K. Hansen (March, 2002) Induction of Hepatocyte Proliferation in vitro by Inhibition of Cell Cycle Inhibitors. U.S. Patent Application (Provisional).
38. Tzanakakis, E. S., C. C. Hsiao, T. Matusushita, R. P. Remmel and W. S. Hu (2001) Probing enhanced cytochrome P450 2B½ activity in rat hepatocyte spheorids through confocal laser scanning microscopy. Cell Transp. 10: 329-42.
39. Bender, V., S. Buschlen and D. Cassio (1998) Expression and localization of hepatocyte domain-specific plasma membrane proteins in hepatoma x fibroblast hybrids and in hepatoma dedifferentiated variants. J. Cell Sci. 111: 3437.
40. Koivisto, U. M., A. L. Hubbard and I. A. Mellman (2001) A novel cellular phenotype for familial hypercholesterolemia due to a defect polarized targeting of LDL receptor. Cell 105: 575-85.
41. Chen, A., M. S. Kinch, T. H. Lin, K. Burridge and R. L. Juliano (1994) Integrin-mediated cell adhesion activates mitogen-activated protein kinases. J. Biol. Chem. 269: 26602-5.
42. Morino, N., T. Mimura, K. Hamasaki, K. Tobe, K. Ueki, K. Kikuchi, K. Takehara, T. Kadowaki, Y. Yazaki and Y. Nojima (1995) Matrix/integrin interaction activates the mitogen-activated protein kinase, p44erk-1 and p42erk-2. J. Biol. Chem. 271: 269-73.
43. Zhu, X. and R. K. Assoian (1995) Integrin-dependent activation of MAP kinase: A link to shape-dependent cell proliferation. Mol. Biol. Cell 6: 273-82.
44. Fassett, J. T., D. Tobolt, C. J. Nelsen, J. H. Albrecht and L. K. Hansen (2002) The role of collagen structure in mitogen stimulation of ERK, cyclin D1 expression, and G1-S progression in rat hepatocytes. J. Biol. Chem. (accepted for publication):
45. Dunn, J. C. Y., M. L. Yarmush, H. G. Koebe and R. G. Tompkins (1989) Hepatocyte function and extracellular matrix geometry: Long-term culture in a sandwich configuration. FASEB J. 3: 174-7.
46. Dunn, J. C., R. G. Tompkins and M. L. Yarmush (1992) Hepatocytes in collagen sandwich: evidence for transcriptional and translational regulation. J. Cell Biol. 115: 1043-53.
47. Howe, A. K. and R. L. Juliano (2000) Regulation of anchorage-dependent signal transduction by protein kinase A and p21-activated kinase. Nature Cell Biol. 2: 593-600.
48. Day, R. N., J. A. Walder and R. A. Maurer (1989) A protein kinase inhibitor gene reduces both basal and multihormone-stimulated prolactin gene transcription. J. Biol. Chem. 264: 431-6.
49. Cascio, S. and K. S. Zaret (1991) Hepatocyte differentiation initiates during endodermal-mesenchymal interactions prior to liver formation. Development 113: 217-25.
50. Saso, K., G. Moehren, K. Higashi and J. B. Hoek (1997) Differential Inhibition of Epidermal Growth Factor Signaling Pathways in Rat Hepatocytes by Long-term Ethanol Treatment. Gastroent. 112: 2073-88.
51. Vacca, F., A. Bagnoato, K. J. Catt and R. Tecce (2000) Transactivation of the epidermal growth factor receptor in endothelin-1-induced mitogenic signaling in human ovarian carcinoma cells. Cancer Res. 60: 5310-7.
52. Ekanger, R., O. K. Vintermyr, G. Houge, T. E. S. TE, J. D. Scott, E. G. K. EG, T. S. Eikhom, T. Christoffersen, D. Ogreid and S. O. D. SO (1989) The expression of cAMP-dependent protein kinase subunits is differentially regulated during liver regeneration. J. Biol. Chem. 264: 4374-82.
53. Roth, J. S., L. L. Hsieh, C. Peraino and I. B. Weinstein (1990) Isolation of a complementary DNA encoding the catalytic subunit of protein kinase A and studies on the expression of this sequence in rat hepatomas and regenerating liver. Cancer Res. 50: 1675-80.
54. Grieco, D., A. Porcellini, E. V. Avvedimento and M. E. Gottesman (1996) Requirement for cAMP-PKA pathway activation by M phase-promoting factor in the transition from mitosis to interphase. Science 271: 1718-23.
55. Collas, P., K. LeGuellec and K. Tasken (1999) The A-kinase-anchoring protein AKAP95 is a multivalent protein with a key role in chromatin condensation at mitosis. J. Cell Biol. 147: 1167-80.
56. Landsverk, H. B., C. R. Carlson, R. L. Steen, L. Vossebein, F. W. Herberg, K. Tasken and P. Collas (2001) Regulation of anchoring of the RIIalpha regulatory subunit of PKA to AKAP95 by threonine phosphorylation of RIIalpha: Implications for chromosome dynamics at mitosis. J. Cell Sci. 114: 3255-64.
57. Aiken, J., L. Cima, B. Schloo, D. Mooney, L. Johnson, R. Langer and J. P. Vacanti (1990) Studies in rat liver perfusion for optimal harvest of hepatocytes. J. Ped. Surg. 25: 140-5.
58. Hansen, L. K., D. J. Mooney, J. P. Vacanti and D. E. Ingber (1994) Integrin binding and cell spreading on extracellular matrix act at different points in the cell cycle to promote hepatocyte growth. Mol. Biol. Cell 5: 967-75.
59. Albrecht, J. H., B. M. Rieland, C. J. Nelsen and C. L. Ahonen (1999) Regulation of G(1) cyclin-dependent kinases in the liver: role of nuclear localization and p27 sequestration. Am. J. Phys. 277: G1207-16.
60. Yagi, T., J. A. Hardin, Y. M. Valenzuela, H. Miyoshi, G. J. G. GJ and S. L. Nyberg (2001) Caspase inhibition reduces apoptotic death of cryopreserved porcine hepatocytes. Hepatology 33: 1432-40.
61. Nyberg, S. L., K. Shirabe, M. Peshwa, T. D. Sielaff, P. L. Crotty, H. J. Mann, R. P. Remmel, W. D. Payne, W.-S. Hu and F. B. Cerra (1993) Extracorporeal application of a gel-entrapment, bioartificial liver: demonstration of drug metabolism and other biochemical functions. Cell Transpl. 2: 441-52.
62. Sielaff, T., M. Y. Hu, S. Rao, K. Groehler, D. Olson, H. J. Mann, R. P. Remmel, R. A. Shatford, B. Amiot and W.-S. Hu (1995) A technique for porcine hepatocyte harvest and description of differentiated metabolic functions in static culture. Transpl. 59: 1459-63.
Thomson et al., Science 282:1145, 1998
Thomson et al., Proc. Natl. Acad. Sci. USA 92:7844, 1995
Shamblott et al., Proc. Natl. Acad. Sci. USA 95:13726, 1998
Sambrook and Russell, Molecular Cloning: A Laboratory Manual, Cold Spring Harbor
Methods in Enzymology 73B:3 (1981).
Marks et al., New Eng. J. Med. 335:730, 1996,
McGuiness et al., Nature Biotechnol. 14:1449, 1996.
Liang, Peng, et al., Cancer Res. 52:6966, 1992
Schena et al., Science 270:467, 1995;
Eisen et al., Methods Enzymol. 303:179, 1999;
Brown et al., Nat. Genet. 21 Suppl 1:33, 1999
Fritz et al. Science 288:316, 2000;
“Microarray Biochip Technology”, M. Schena ed., Eaton Publishing Company
“Microarray analysis”, Gwynne & Page, Science (Aug. 6, 1999 supplement)
Pollack et al., Nat Genet 23:41, 1999
Gerhold et al., Trends Biochem. Sci. 24:168, 1999
“Gene Chips (DNA Microarrays)”, L Shi, www.Gene-Chips.com.
“In vitro Methods in Pharmaceutical Research”, Academic Press, 1997
Castell et al., pp. 375-410 in “In vitro Methods in Pharmaceutical Research,” Academic Press, 1997
Gomez-Lechon et al. Anal. Biochem. 236:296, 1996
Vickers, “In vitro Methods in Pharmaceutical Research,” pp. 375-410, Academic Press, 1997
Grompe et al. Sem. Liver Dis. 19:7, 1999
Peeters et al., Hepatology 25:884, 1997
Ohashi et al. Nature Med. 6:327, 2000
Braun et al. Nature Med. 6:320, 2000
Rhim et al. Proc. Natl. Acad. Sci. USA 92:4942, 1995
Lieber et al. Proc. Natl. Acad. Sci. USA 92:6210, 1995
Mignon et al. Nature Med. 4:1185, 1998
Overturf et al. Human Gene Ther. 9:295, 1998
Kobayashi et al., Science 287:1258, 2000
Rudolph et al., Science 287:1253, 2000
Macdonald et al., “Cell Encapsulation Technology and Therapeutics”, pp. 252-286,
Kuhtreiber et al. eds., Birkhauser, Boston Mass., 1999

1. A method of inducing proliferative and differentiative mammalian hepatocytes in vitro comprising (a) contacting the hepatocytes with protein kinase A (PKA) inhibitor.

2. The method of claim 1, wherein the protein kinase A inhibitor is a nucleotide or nucleoside derivative of formula I: 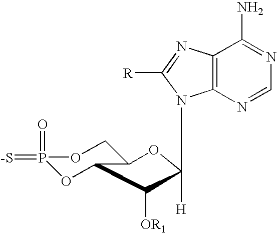
wherein R is hydrogen, halogen, or heterocycloalkyl and R₁ is hydrogen, lower alkyl, or lower acyl.

3. The method of claim 1, wherein the protein kinase A inhibitor is a peptide having SEQ ID NO:2, Xaa-Arg-Arg-Xaa-Ala-Xaa, wherein Xaa is any amino acid.

4. The method of claim 1, wherein the protein kinase A inhibitor is a peptide or polypeptide with SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

5. The method of claim 1, wherein the protein kinase A inhibitor is N-[2-((p-bromocinnamyl)amino)ethyl]-5-isoquinolinesulfonamide.

6. The method of claim 1, wherein the protein kinase A inhibitor is:

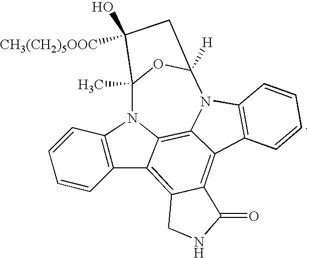

7. The method of claim 1, wherein the protein kinase A inhibitor is:

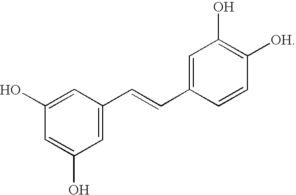

8. The method of claim 1, wherein the protein kinase A inhibitor is: 4,4′,5,5′,6,6′-hexahydroxydiphenic acid 2,6,2′,6′-dilactone, 1-(5-Isoquinolinesulfonyl)-2-methylpiperazine, N-[2-(methylamino)ethyl]-5-isoquinolinesulfonamide, N-(2-aminoethyl)-5-isoquinolinesulfonamide, or (5-isoquinolinesulfonyl)piperazine.

9. The method of claim 1, wherein the hepatocytes are primary hepatocytes.

10. The method of claim 1, wherein the hepatocytes are generated from stem cells.

11. The method of claim 1, comprising (b) growing the hepatocytes on a biocompatible support matrix.

12. The method of claim 11, wherein the support matrix is a collagen gel matrix.

13. The method of claim 11, wherein the support matrix is a collagen film.

14. The method of claim 11, wherein the solid substrate is a collagen gel sandwich.

15. The method of claim 11, further comprising (c) growing the hepatocytes in the absence of PKA inhibitor for an interval of time.

16. The method of claim 15, further comprising (d) repeating steps (a) and (b).

17. The method of claim 1, wherein the hepatocytes are of human origin.

18. The method of claim 1, wherein the hepatocytes are of porcine origin.

19. The method of claim 1, wherein the protein kinase A inhibitor is present at a concentration of 0. 1, 0.5, 1, 3, or 5 μM.

20. The method of claim 1, wherein the protein kinase A inhibitor is present at a concentration of about 1 to 3 μM.

21. A method of promoting survival of differentiative mammalian hepatocytes in vitro comprising contacting the hepatocytes with protein kinase A inhibitor.

22. A method of screening a compound for its effect on hepatocytes or a hepatocyte activity, comprising:
(a) combining the compound with a cell population obtained by treating hepatocytes with a protein kinase A;
(b) determining any change to cells in the population or their activity that results from being combined with the compound; and
(c) correlating the change with the effect of the compound on hepatocytes or a hepatocyte.

23. The method of claim 22, comprising determining whether the compound is toxic to cells in the population.

24. The method of claim 22, comprising determining whether the compound affects cell proliferation in the population or maintenance in culture of cells in the population.

25. The method of claim 22, comprising determining whether the compound changes enzyme activity or secretion.

26. The method of claim 25 comprising determining whether the compound changes activity of a hepatocyte Phase I metabolizing enzyme.

27. The method of claim 25, comprising determining whether the compound changes activity of a hepatocyte Phase II metabolizing enzyme.

28. The method of claim 25, comprising determining whether the compound changes cytochrome p450 expression or activity.

29. The method of claim 25, comprising determining whether the compound changes CYP3A3-5 activity, CYP2D activity, or CYP2C9 activity.

30. The method of claim 25, comprising determining whether the compound affects CYP1A1 or CYP1A2 activity.

31. The method of claim 22, comprising determining whether the compound affects the activity of 7-ethoxycoumarin O-de-ethylase, aloxyresorufin O-de-alkylase, coumarin 7-hydroxylase, p-nitrophenol hydroxylase, testosterone hydroxylation, UDP-glucuronyltransferase, glutathione S-transferase, gamma-glutamyl tranpeptidase, or glucose-6-phosphatase.

32. The method of claim 22, comprising determining whether the compound affects the synthesis of a plasma protein.

33. The method of claim 32, wherein the plasma protein is albumin, transferrin, alpha₁-antitrypsin, or alpha-fetoprotein.

34. The method of claim 22, comprising determining whether the compound affects gluconeogenesis, ureagenesis, bilirubin conjugation, or bile acid conjugation.

35. The method of claim 22, comprising determining whether the compound affects synthesis or secretion of cholesterol or lipoprotein, levels of glutathione, nucleoside phosphate metabolism, intracellular K²⁺ or Ca⁺ concentration, release of nuclear matrix proteins or oligonucleosomes, induction of apoptosis, or glycogen storage.

36. The method of claim 22, wherein the hepatocytes are human in origin.

37. The method of claim 22, wherein cells in the population have been genetically altered.

38. A bioartificial liver device comprising a proliferative and differentiative mammalian hepatocyte.

39. The bioartificial liver device of claim 38, further comprising a biocompatible support matrix.

40. The device of claim 39, wherein the support matrix is a collagen gel matrix.

41. The device of claim 39, wherein the support matrix is a collagen film.

42. The device of claim 39, wherein the support matrix is a collagen gel sandwich.

43. An artificial liver device comprising:
(a) a cell culture layer comprising cells from an isolated cell line of normal hepatocytes and a protein kinase A inhibitor, and
(b) a support matrix that provides means for fluid circulation across the cell culture layer.

44. The artificial liver device of claim 43, wherein the fluids are culture medium, plasma or blood.

45. A method of transplanting hepatocytes comprising:
(a) inducing a population of proliferative and differentiative mammalian hepatocytes in vitro comprising contacting the hepatocytes with protein kinase A inhibitor; and
(b) transplanting the population of hepatocytes.
